(19) United States
(12) Patent Application Publication
(10) Pub. No.: US 2009/0130029 A1
(43) Pub. Date: May 21, 2009

(54) GLYCEROL ETHERS VEHICLE AND PHARMACEUTICAL COMPOSITIONS THEREOF

(75) Inventors: Dov TAMARKIN, Maccabim (IL); Alex Besonov, Rehovot (IL)

Correspondence Address: WILMERHALE/BOSTON, 60 STATE STREET, BOSTON, MA, 02109, US

(73) Assignee: Foamix Ltd., Ness Ziona (IL)

(21) Appl. No.: 12/275,532

(22) Filed: Nov. 21, 2008

Related U.S. Application Data
(60) Provisional application No. 60/989,738, filed on Nov. 21, 2007.

(51) Int. Cl. A61K 9/12

(52) U.S. Cl. 424/47

(57) ABSTRACT

The present disclosure teaches unique foamable vehicles or carriers comprising at least one glycerol ether, a stabilizer, and water; pharmaceutical and cosmetic compositions with potentially enhanced skin delivery and their uses.

### CROSS-REFERENCE TO RELATED APPLICATIONS

This application claims priority to U.S. Application Ser. No. 60/989,738, filed on Nov. 21, 2007, which is herein incorporated by reference in its entirety.

### FIELD OF THE INVENTION

This disclosure relates to foamable pharmaceutical and cosmetic compositions.

### BACKGROUND

External topical administration is an important route for the administration of drugs in disease treatment. Many groups of drugs, including, for example, antibiotic, anti-fungal, anti-inflammatory, anesthetic, analgesic, anti-allergic, corticosteroid, retinoid and anti-proliferative medications are preferably administered in hydrophobic media, namely ointment. However, ointments often form an impermeable barrier, so that metabolic products and excreta from the wounds to which they are applied are not easily removed or drained away. Furthermore, it is difficult for the active drug dissolved in the carrier to pass through the white petrolatum barrier layer into the wound tissue, so the efficacy of the drug is reduced. In addition, ointments and creams often do not create an environment for promoting respiration of the wound tissue and it is not favorable to the normal respiration of the skin. An additional disadvantage of petroleum jelly-based ointments and creams relates to the greasy feeling left following their topical application onto the skin, mucosal membranes and wounds.

Foams are considered a more convenient vehicle for topical delivery of active agents. There are several types of topical foams, including aqueous foams, such as commonly available shaving foams; hydroalcoholic foams, emulsion-based foams, comprising oil and water components, and oleaginous foams, which consist of high oil content. In skin therapy, oil containing foams are preferred, since oil contributes to skin protection and moisturization, which improve the therapeutic effect of the formulation.

Volatile ether foam formulations are novel and are investigated herein. By virtue of their volatile nature they present a unique technical challenge to provide foam formulations that are thermolabile, easily breakable upon mechanical stimulation and remain stable although being thermolabile for sufficient period of time to deliver a cosmetic or pharmaceutical agent topically to the skin.

By virtue of their volatile nature if foam formulations are left for a period of time the formulation will evaporate with the non-volatile components being deposited and/or absorbed on the skin or topical surface. Some of the technical challenges of such formulations include how to overcome the manufacturing and storage problems due to the volatility of the system, how to control the disappearance rate of the ethers so that the foam produced is stable for a sufficient period of time that it can be conveniently applied and spread on a topical area and to provide a compatible non-volatile second solvent preferably to facilitate delivery of any active during and after evaporation of the volatile ether.

There remains an unmet need for, easy to use, vehicles and foam formulations, comprising volatile ethers, and ether like substances, counterparts and derivatives thereof and preferably in substantial or higher concentrations which can effectively deliver and/or deposit various active and cosmetic agents into and onto the skin and/or other target sites and are relatively non-irritating and thus suitable for use by people having sensitive skin, body cavities and mucosal surfaces.

### SUMMARY

The present disclosure relates to volatile ether and ether like substances, counterparts and derivatives thereof carrier compositions, and foams and pharmaceutical and cosmetic compositions containing them.

In some embodiments of the present disclosure there is provided a pharmaceutical or cosmetic vehicle composition comprising: (a) less than or equal to about 75% by weight of at least one glycerol ether; (b) at least one stabilizer selected from the group consisting of a surface-active agent, a polymeric agent and mixtures thereof; (c) water; and (d) about 3% to about 35% by weight of a liquefied hydrocarbon gas propellant; wherein the polymeric agent, if present, is about 0.1% to about 5% by weight and is selected from the group consisting of a bioadhesive agent, a gelling agent, a film forming agent and a phase change agent; wherein the composition is contained in a pressurized container; and wherein the composition is substantially flowable and provides a breakable and thermolabile foam of fairly good to excellent quality upon release from the container.

In some embodiments the composition further comprises a carrier or solvent selected from the group consisting of an organic carrier, a hydrophilic solvent, a hydrophobic solvent, a potent solvent, a polar solvent, a silicone, an emollient, a liquid wax and mixtures thereof; wherein the organic carrier is selected from the group consisting of mineral oil, a therapeutic oil, triglycerides, medium chain triglyceride (MCT) oil, capric/caprylic triglyceride, cocoglycerides, alkyl esters of fatty acids such as isopropyl palmitate, isopropyl myristate, isopropyl isostearate, poly propylene glycol 15-stearyl ether, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, maleated soybean oil, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxane, polyaryl siloxane, polyalkylaryl siloxane, a polyether siloxane copolymer and a poly(dimethylsiloxane)-(diphenyl-siloxane) copolymer and a polypropylene glycol alkyl ether; wherein the polar solvent is selected from the group consisting of dimethyl isosorbide, glycerol, propylene glycol, hexylene glycol, diethylene glycol, propylene glycol n-alkanols, terpenes, di-terpenes, tri-terpenes, limonene, terpene-ol, 1-menthol, dioxolane, ethylene glycol, other glycols, oleyl alcohol, alpha-hydroxy acids, such as lactic acid and glycolic acid, sulfoxides, such as dimethylsulfoxide (DMSO), dimethylformanide, methyl dodecyl sulfoxide, dimethylacetamide, azone (1-dodecylazacycloheptan-2-one), 2-(n-nonyl)-1,3-dioxolane, alkanols, such as dialkylamino acetates, and admixtures thereof; wherein the silicone is selected from the group consisting of a volatile silicone and non-volatile silicone; and wherein the liquid wax is selected from the group consisting of isostearic acid, oleyl alcohol, and capric alcohol, capryl alcohol, isostearic acid, caprylic acid, caproic acid, and butyric acid, jojoba oil.

In some embodiments of the composition the surface-active agent is a liquid or a combination comprising a solid and a liquid. In some embodiments of the composition the surface active agent is selected from the group consisting of a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59, a polyoxyethylene alkylyl ether, polyoxyethylene cetyl ether, polyoxyethylene palmityl ether, polyethylene oxide hexadecyl ether, polyethylene glycol cetyl ether, brij 38, brij 52, brij 56 and brij W1, a sucrose ester, a partial ester of sorbitol, sorbitan monolaurate, sorbitan monolaurate a monoglyceride, a diglyceride, isoceteth-20, a sucrose ester, or selected from the group consisting of steareth 2, glyceryl monostearate/PEG 100 stearate, Glyceryl Stearate, Steareth-21, peg 40 stearate, polysorbate 40, polysorbate 60, polysorbate 80, sorbitan stearate, laureth 4, Sorbitan monooleate, ceteareth 20, steareth 20, ceteth 20, Macrogol Cetostearyl Ether, ceteth 2, PEG-30 Dipolyhydroxystearate, sucrose distearate, polyoxyethylene (100) stearate, PEG 100 stearate, PEG 40 stearate, laureth 4, cetomacrogol ether, Cetearyl alcohol, Cetearyl glucoside, Oleyl alcohol, Steareth-2, Diisopropyl adipate, Capric/caprilic triglicerides, Polysorbate 20; Montanov 68 (cetearyl alcohol and cetearyl glucoside), Sharonmix 824 (a liquid blend of methyl paraben, ethyl paraben and propyl paraben—in phenoxyethanol), Simusol 165 (Glyceryl stearate and PEG-100 stearate), methyl glucose sequistearate, PEG 30 dipolyhydroxystearate, sucrose stearic acid esters, sorbitan laureth, sorbitan stearate and mixtures thereof.

In some embodiments of the composition the polymeric agent is selected from the group consisting of avicel, carbopol 934, pemulen TR2, klucel EF, xanthan gum, methocel A4M, carboxy methyl cellulose, locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, an amine-bearing polymer, chitosan, alginic acid, hyaluronic acid, a chemically modified starch, a carboxyvinyl polymer, polyvinylpyrrolidone, polyvinyl alcohol, a polyacrylic acid polymer, a polymethacrylic acid polymer, polyvinyl acetate, a polyvinyl chloride polymer, a polyvinylidene chloride polymer, methylcellulose, hydroxypropyl cellulose, hydroxypropyl methylcellulose, hydroxyethyl cellulose, hydroxy propylmethyl cellulose, methylhydroxyethylcellulose, methyl hydroxypropylcellulose, hydroxyethylcarboxymethylcellulose, carboxymethyl cellulose, carboxymethylcellulose carboxymethylhydroxyethylcellulose, a cationic cellulose, PEG 1000, PEG 4000, PEG 6000, PEG 8000, a derivatized polymeric emulsifier, aluminum starch octenylsuccinate (ASOS), sodium starch octenylsuccinate.

In some embodiments of the composition the glycerol ether is a volatile glycerol ether, and wherein the glycerol ether evaporates upon application to a topical surface at about 37° C. In some embodiments of the composition the glycerol ether is selected from the group consisting of glycerol propyl ether, glycerol hexyl ether, glycerol nonyl ether and mixtures thereof.

In some embodiments the composition contains a non-volatile ether. In some embodiments the non-volatile ether is selected from the group consisting of Glycerol, 1(2-chlorophenyl)ether; glycerol, 1-(4-chlorophenyl)ether; glycerol, 1,2-dimethyl ether; glycerol, 1,3-dimethyl ether; glycerol, 1,3-diphenylether; glycerol, 1-(2-methoxyphenyl)ether; glycerol, 1-methyl ether; glycerol, 2-methyl ether; glycerol, 1-octadecyl ether; glycerol, 1-phenyl ether; and polyoxypropylene glyceryl ether.

In some embodiments of the composition further comprises a foam adjuvant.

In some embodiments of the composition the ratio of glycerol ether to stabilizer and water ranges from about 9:1 to about 1:100. In some embodiments of the composition the ratio of glycerol ether to stabilizer and water ranges from about 5:1 to about 1:2.

In some embodiments the composition further comprises an additional component selected from the group consisting of a modulating agent, a polar solvent, an antiperspirant, an anti-static [0009] agent, a buffering agent, a bulking agent, a chelating agent, a colorant, a conditioner, a deodorant, a diluent, a dye, an emollient, fragrance, a humectant, an occlusive agent, a penetration enhancer, a perfuming agent, a permeation enhancer, a pH-adjusting agent, a preservative, a skin penetration enhancer, a sunscreen, a sun blocking agent, a sunless tanning agent, an antioxidant, an antiseptive, a flavonoid, and a vitamin.

In some embodiments the composition is in a non-foam state.

In some embodiments of the present disclosure there is provided a therapeutic composition comprising: i. at least one volatile glycerol ether; ii. at least one stabilizer selected from the group consisting of a surface-active agent, a polymeric agent and mixtures thereof; iii. water; iv. a pharmaceutical or cosmetic agent homogeneously distributed in the composition; and v. about 3% to about 35% by weight of a liquefied hydrocarbon gas propellant; wherein the polymeric agent, if present is about 0.1% to about 5% by weight and is selected from the group consisting of a bioadhesive agent, a gelling agent, a film forming agent and a phase change agent; wherein the volatile glycerol ether evaporates upon application to a topical surface at about 37° C. to deposit the pharmaceutical or cosmetic agent on the target topical surface; and wherein the composition is contained in a pressurized container; and wherein the composition is substantially flowable and provides a breakable and thermolabile foam of fairly good to excellent quality upon release from the container.

In some embodiments of the composition, the pharmaceutical or cosmetic agent is selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, coal tar, dicarboxylic acids, disinfectants, fungicides, hair growth regulators, hormones, hydroxy acids, interferons, immunosuppressants, immunoregulating agents, insecticides, insect repellents, keratolytic agents, lactams, metals, metal oxides, mitocides, neuropeptides, steroids, non-steroidal anti-inflammatory agents, oxidizing agents, pediculicides, photodynamic therapy agents, phosphcholines, alkyl phosphoric acids, retinoids, sanatives, scabicides, self tanning agents, skin whitening agents, vasoconstrictors, vasodilators, vitamins, vitamin D derivatives, wound healing agents and wart removers as well as agents having activity against skin and topical lesions, leishmaniasis, superficial basal cell carcinomas, actinic keratoses, Bowen's disease and/or other squamous cell carcinomas and molluscum contagiosum.

In some embodiments of the composition, the volatile glycerol ether is present in the composition in an amount sufficient to solubilize the active agent.

In some embodiments of the composition, the active agent is a phosphochline or an alkyl phosphoric acid. In some embodiments of the composition, the active agent is hexadecylphosphocholine. In some embodiments of the composition, the active agent is effective against any one or more of skin or topical lesions, cancer, skin cancer and leishmaniasis.

In some embodiments the composition further comprises at least one excipient compound selected from the group consisting of a petrolatum, a therapeutic oil, a liquid wax, a liquid emollient and mixtures thereof.

In some embodiments the composition further comprises a carrier or solvent selected from the group consisting of an organic carrier, a hydrophilic solvent; a hydrophobic solvent; a potent solvent; a polar solvent, a silicone, an emollient, a liquid wax and mixtures thereof.

In some embodiments of the composition, the glycerol ether, stabilizer and water are selected to generate a single phase formulation or substantially homogenous suspension. In some embodiments of the composition, the glycerol ether, stabilizer and water are selected to generate an emulsion that is substantially resistant to phase reversal.

In some embodiments of the present disclosure there is provided a method of treating, ameliorating or preventing a disorder of a mammalian subject, the method comprising: administering a foamable therapeutic composition to a target site, the composition comprising: a. a therapeutically effective amount of a pharmaceutical or cosmetic agent substantially homogenously distributed in the glycerol ether composition; b. less than or equal to about 75% by weight of at least one glycerol ether; c. at least one stabilizer selected from the group consisting of a surface-active agent, a polymeric agent and mixtures thereof; d. about 3% to about 35% by weight of a liquefied hydrocarbon gas propellant; and wherein the polymeric agent is about 0.1% to about 5% by weight and is selected from the group consisting of a bioadhesive agent, a gelling agent, a film forming agent and a phase change agent; wherein the composition is contained in a pressurized container; and wherein the composition is substantially flowable and provides a breakable and thermolabile foam of fairly good to excellent quality upon release from the container; and wherein the glycerol ether evaporates upon application to a topical surface at about 37° C. to deposit the pharmaceutical or cosmetic agent on the target topical surface.

In some embodiments, the target site is selected from the group consisting of the skin, a body cavity, a mucosal surface, the ear canal, the vagina and the rectum.

In some embodiments, the disorder is selected from the group consisting of dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, eethyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, granuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo; and wherein the active agent is suitable for treating said disorderm or is selected from the group consisting of chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranuloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, topical or skin lesions, leishmaniosis, cancer, skin cancer, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; warts; herpes simplex virus infections, other viral infections, skin and topical lesions, leishmaniasis, superficial basal cell carcinomas, actinic keratoses, Bowen's disease and/or other squamous cell carcinomas, molluscum contagiosum and eczema, and wherein the active agent is suitable for treating said disorder.

In some embodiments, the disorder is a dermatological disorder which can be treated, ameliorated or prevented by a topical phosphocholine or alkyl phosphoric acid.

In some embodiments, the active agent is selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, coal tar, dicarboxylic acids, disinfectants, fungicides, hair growth regulators, hormones, hydroxy acids, interferons, immunosuppressants, immunoregulating agents, insecticides, insect repellents, keratolytic agents, lactams, metals, metal oxides, mitocides, neuropeptides, steroids, non-steroidal anti-inflammatory agents, oxidizing agents, pediculicides, photodynamic therapy agents, phosphcholines, alkyl phosphoric acids, retinoids, sanatives, scabicides, self tanning agents, skin whitening agents, vasoconstrictors, vasodilators, vitamins, vitamin D derivatives, wound healing agents and wart removers as well as agents having activity against skin and topical lesions, leishmaniasis, superficial basal cell carcinomas, actinic keratoses, Bowen's disease and/or other squamous cell carcinomas and molluscum contagiosum.

In some embodiments, the glycerol ether is a volatile glycerol ether, wherein the volatile glycerol ether evaporates upon application to a topical surface at about 37° C.

In some embodiments of the present disclosure there is provided a pharmaceutical composition comprising: i. a liquid volatile glycerol ether; ii. a stabilizer comprising about 1% to about 5% of at least one surface-active agent and about 0.1% to about 5% of at least one polymeric agent selected from the group consisting of a bioadhesive agent, a gelling agent, a film forming agent and a phase change agent; iii. water; iv. an active agent substantially homogenously distributed in the composition; and v. 3% to about 35% by weight of a liquefied hydrocarbon gas propellant wherein the volatile glycerol ether evaporates upon application to a topical surface at about 37° C. to deposit the pharmaceutical or cosmetic agent for absorption onto a topical surface; and wherein the composition is contained in a pressurized container; and wherein the composition provides a breakable and thermolabile foam of about fairly good to about excellent quality upon release from the container.

In some embodiments, a liquid volatile glycerol ether comprises about 1% to about 75% by weight of a volatile glycerol ether selected from the group consisting of glycerol propyl ether, glycerol hexyl ether and glycerol nonyl ether and mixtures thereof.

In some embodiments of the present disclosure there is provided a pharmaceutical or cosmetic vehicle composition comprising: (a) less than or equal to about 75% by weight of at least one glycerol ether; (b) at least one stabilizer selected from the group consisting of a surface-active agent, a polymeric agent and mixtures thereof; and (c) water; wherein the polymeric agent, if present, is about 0.1% to about 10% by weight of and is selected from the group consisting of a bioadhesive agent, a gelling agent, a film forming agent and a phase change agent; wherein the composition is contained in a pressurized container; and wherein the composition is substantially flowable and provides a breakable and thermolabile foam of about fairly good to about excellent quality upon release from the container; and wherein the composition is able to deposit a pharmaceutical or cosmetic agent for absorption onto a topical surface.

There is also provided a formulation of any of the compositions described above for use in the manufacture of or as a medicament.

### DETAILED DESCRIPTION

The present disclosure relates to an aqueous composition comprising one or more volatile ethers alone or in combination with non-volatile ethers for use as vehicle, therapeutic, cosmetic or pharmaceutical composition.

In accordance with one or more embodiments there is provided a pharmaceutical or cosmetic vehicle composition comprising:
(a) at least one glycerol ether;
(b) at least one stabilizer selected from the group consisting of a surface-active agent, a polymeric agent and mixtures thereof; and
(c) water;

wherein the polymeric agent is about 0.01% to about 10% by weight and is selected from the group consisting of a bioadhesive agent, a gelling agent, a film forming agent and a phase change agent;

wherein the glycerol ether composition is able to deposit a pharmaceutical or cosmetic agent for absorption onto the topical surface; and

wherein the composition is contained in a pressurized container and further comprises a liquefied hydrocarbon gas propellant at a concentration of about 3% to about 35% by weight of the total composition it is substantially flowable and provides a breakable and thermolabile foam of about fairly good to about excellent quality upon release.

In one or more embodiments the at least one glycerol ether is volatile and evaporates upon application of the foam to a topical surface and is able to deposit a pharmaceutical or cosmetic agent for absorption onto the topical surface;

In an embodiment the glycerol ether is at a concentration of about or less than about 75 wt %.

In an embodiment the composition further comprises a carrier or solvent selected from the group consisting of an organic carrier, a hydrophilic solvent, a hydrophobic solvent, a potent solvent, a polar solvent, a silicone, an emollient, a liquid wax and mixtures thereof.

In an embodiment the composition is substantially resistant to one or more Freeze-Thaw cycles (FTC).

In one or more embodiments the surface-active agent is a liquid or a solid and a liquid.

In one or more embodiments the surface active agent is selected from the group consisting of a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59, a polyoxyethylene alkylyl ether, polyoxyethylene cetyl ether, polyoxyethylene palmityl ether, polyethylene oxide hexadecyl ether, polyethylene glycol cetyl ether, brij 38, brij 52, brij 56 and brij W1, a sucrose ester, a partial ester of sorbitol, sorbitan monolaurate, sorbitan monolaurate a monoglyceride, a diglyceride, isoceteth-20, a sucrose ester, or selected from the group consisting of steareth 2, glyceryl monostearate/PEG 100 stearate, Glyceryl Stearate, Steareth-21, peg 40 stearate, polysorbate 40, polysorbate 60, polysorbate 80, sorbitan stearate, laureth 4, Sorbitan monooleate, ceteareth 20, steareth 20, ceteth 20, Macrogol Cetostearyl Ether, ceteth 2, PEG-30 Dipolyhydroxystearate, sucrose distearate, polyoxyethylene (100) stearate, PEG 100 stearate, PEG 40 stearate, laureth 4, cetomacrogol ether, Cetearyl alcohol, Cetearyl glucoside, Oleyl alcohol, Steareth-2, Diisopropyl adipate, Capric/caprilic triglicerides, Polysorbate 20; Montanov 68 (cetearyl alcohol (and) cetearyl glucoside.), Sharonmix 824 (a liquid blend of methyl paraben, ethyl paraben and propyl paraben—in phenoxyethanol), Simusol 165 (Glyceryl stearate and PEG-100 stearate). Methyl glucose sequistearate, Peg 30 dipolyhydroxystearate, sucrose stearic acid esters, sorbitan laureth, sorbitan stearate and mixtures thereof.

In one or more embodiments the polymeric agent is selected from the group consisting of avicel, avicel 581RC, carbopol, carbopol 934, pemulen, pemulen TR2, klucel EF, xanthan gum, methocel A4M, carboxy methyl cellulose, locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, an amine-bearing polymer, chitosan, alginic acid, hyaluronic acid, a chemically modified starch, a carboxyvinyl polymer, polyvinylpyrrolidone, polyvinyl alcohol, a polyacrylic acid polymer, a polymethacrylic acid polymer, polyvinyl acetate, a polyvinyl chloride polymer, a polyvinylidene chloride polymer, methylcellulose, hydroxypropyl cellulose, hydroxypropyl methylcellulose, hydroxyethyl cellulose, hydroxy propylmethyl cellulose, methylhydroxyethylcellulose, methyl hydroxypropylcellulose, hydroxyethylcarboxymethylcellulose, carboxymethyl cellulose, carboxymethylcellulose carboxymethylhydroxyethylcellulose, a cationic cellulose, microcrystalline cellulose, PEG 1000, PEG 4000, PEG 6000, PEG 8000, a derivatized polymeric emulsifier, aluminum starch octenylsuccinate (ASOS), sodium starch octenylsuccinate.

In one or more embodiments the glycerol ether is selected from the group consisting of a volatile or a non-volatile glycerol ether.

In one or more embodiments the glycerol ether is selected from the group consisting of glycerol propyl ether, glycerol hexyl ether, glycerol nonyl ether and mixtures thereof.

In one or more embodiments the composition further comprising an active agent.

In one or more embodiments the active agent is selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, coal tar, dicarboxylic acids, disinfectants, fungicides, hair growth regulators, hormones, hydroxy acids, interferons, immunosuppressants, immunoregulating agents, insecticides, insect repellents, keratolytic agents, lactams, metals, metal oxides, mitocides, neuropeptides, steroids, non-steroidal anti-inflammatory agents, oxidizing agents, pediculicides, photodynamic therapy agents, phosphcholines, alkyl phosphoric acids, retinoids, sanatives, scabicides, self tanning agents, skin whitening agents, vasoconstrictors, vasodilators, vitamins, vitamin D derivatives, wound healing agents and wart removers as well as agents having activity against superficial basal cell carcinomas, actinic keratoses, Bowen's disease and/or other squamous cell carcinomas and molluscum contagiosum.

In one or more embodiments the composition further contains a non-volatile ether.

In one or more embodiments the non-volatile ether is selected from the group consisting of Glycerol, 1(2-chlorophenyl)ether; glycerol, 1-(4-chlorophenyl)ether; glycerol, 1,2-dimethyl ether; glycerol, 1,3-dimethyl ether; glycerol, 1,3-diphenylether; glycerol, 1-(2-methoxyphenyl)ether; glycerol, 1-methyl ether; glycerol, 2-methyl ether; glycerol, 1-octadecyl ether; glycerol, 1-phenyl ether; and polyoxypropylene glyceryl ether.

In one or more embodiments the composition further contains a foam adjuvant

In one or more embodiments the organic carrier is selected from the group consisting of mineral oil, a therapeutic oil, triglycerides, medium chain triglyceride (MCT) oil, capric/caprylic triglyceride, cocoglycerides, alkyl esters of fatty acids such as isopropyl palmitate, isopropyl myristate, isopropyl isostearate, poly propylene glycol 15-stearly ether, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, maleated soybean oil, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxane, polyaryl siloxane, polyalkylaryl siloxane, a polyether siloxane copolymer and a poly(dimethylsiloxane)-(diphenyl-siloxane) copolymer and a polypropylene glycol alkyl ether.

In one or more embodiments the polar solvent is selected from the group consisting of dimethyl isosorbide, glycerol, propylene glycol, hexylene glycol, diethylene glycol, propylene glycol n-alkanols, terpenes, di-terpenes, tri-terpenes, limonene, terpene-ol, l-menthol, dioxolane, ethylene glycol, other glycols, oleyl alcohol, alpha-hydroxy acids, such as lactic acid and glycolic acid, sulfoxides, such as dimethylsulfoxide (DMSO), dimethylformanide, methyl dodecyl sulfoxide, dimethylacetamide, azone (1-dodecylazacycloheptan-2-one), 2-(n-nonyl)-1,3-dioxolane, alkanols, such as dialkylamino acetates, and admixtures thereof.

In one or more embodiments the silicone is selected from the group consisting of a volatile silicone and non-volatile silicone.

In one or more embodiments the emollient is cetearyl alcohol.

In one or more embodiments the liquid wax is selected from the group consisting of isostearic acid, oleyl alcohol, and capric alcohol, capryl alcohol, isostearic acid, caprylic acid, caproic acid, and butyric acid, jojoba oil.

In one or more embodiments the ratio of glycerol ether to stabilizer and water ranges from about 9:1 to about 1:100.

In one or more embodiments the ratio of glycerol ether to stabilizer and water ranges from about 5:1 to about 1:2.

In one or more embodiments the composition further comprises an additional component selected from the group consisting of a modulating agent, a polar solvent, an antiperspirant, an anti-static agent, a buffering agent, a bulking agent, a chelating agent, a colorant, a conditioner, a deodorant, a diluent, a dye, an emollient, fragrance, a humectant, an occlusive agent, a penetration enhancer, a perfuming agent, a permeation enhancer, a pH-adjusting agent, a preservative, a skin penetration enhancer, a sunscreen, a sun blocking agent, a sunless tanning agent, an antioxidant, an antiseptive, a flavanoid, and a vitamin.

In accordance with one or more embodiments there is provided a therapeutic composition comprising:
i. at least one volatile glycerol ether;
ii. at least one stabilizer selected from the group consisting of a surface-active agent, a polymeric agent and mixtures thereof;
iii. water; and
iv. an active agent;

wherein the polymeric agent is about 0.01% to about 5% by weight and is selected from the group consisting of a bioadhesive agent, a gelling agent, a film forming agent and a phase change agent;

wherein the volatile glycerol ether evaporates upon application to a topical surface and is able to deposit a pharmaceutical or cosmetic agent for absorption onto the topical surface; and

wherein the composition is contained in a pressurized container and further comprises a liquefied hydrocarbon gas propellant at a concentration of about 3% to about 35% by weight of the total composition it is substantially flowable and provides a breakable thermolabile foam of about fairly good to about excellent quality upon release.

In one or more embodiments the volatile glycerol ether is present in the composition in an amount sufficient to solubilize the active agent.

In one or more embodiments the active agent is a phosphocholine or an alkyl phosphoric acid.

In one or more embodiments the active agent is hexadecylphosphocholine.

In one or more embodiments the active agent is effective against any one or more of skin or topical lesions, cancer, skin cancer and leishmaniasis.

In one or more embodiments the alkyl phosphoric acid or phosphocholine is used in combination with at least one excipient compound selected from the group consisting of a petrolatum, a therapeutic oil, a liquid wax, a liquid emollient and mixtures thereof.

In one or more embodiments the glycerol ether, stabilizer and water are selected to generate a single phase formulation or homogeneous suspension.

In one or more embodiments the glycerol ether, stabilizer and water are selected to generate an emulsion that is substantially resistant to phase reversal.

In one or more embodiments there is provided a method of treating, ameliorating or preventing a disorder of a mammalian subject, comprising:

administering a foamable therapeutic composition to a target site, the composition comprising:

a. a therapeutically effective amount of an active agent;
b. at least one glycerol ether;
c. at least one stabilizer selected from the group consisting of a surface-active agent, a polymeric agent and mixtures thereof; and

wherein the polymeric agent is about 0.01% to about 5% by weight and is selected from the group consisting of a bioadhesive agent, a gelling agent, a film forming agent and a phase change agent;

wherein the glycerol ether composition is able to deposit a pharmaceutical or cosmetic agent for absorption onto the topical surface; and

wherein the composition is contained in a pressurized container and further comprises a liquefied hydrocarbon gas propellant at a concentration of about 3% to about 35% by weight of the total composition it is substantially flowable and provides a breakable thermolabile foam of about fairly good to about excellent quality upon release.

In one or more embodiments the target site is selected from the group consisting of the skin, a body cavity, a mucosal surface, the, the ear canal, the vagina and the rectum.

In one or more embodiments the disorder is selected from the group consisting of dermatological pain, dermatological inflammation, acne, acne vulgaris, inflammatory acne, non-inflammatory acne, acne fulminans, nodular papulopustular acne, acne conglobata, dermatitis, atopic dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, eethyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, granuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo; and wherein the active agent is suitable for treating said disorderm or is selected from the group consisting of chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranuloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, topical or skin lesions, leishmaniosis, cancer, skin cancer, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; warts; herpes simplex virus infections, other viral infections, skin and topical lesions, leishmaniasis, superficial basal cell carcinomas, actinic keratoses, Bowen's disease and/or other squamous cell carcinomas, molluscum contagiosum and eczema, and wherein the active agent is suitable for treating said disorder.

In one or more embodiments the disorder is a dermatological disorder, which can be treated, ameliorated or prevented by a topical phosphocholine oralkyl phosphoric acid.

In one or more embodiments there is provided a pharmaceutical composition comprising:

i. a liquid volatile glycerol ether;

ii. a stabilizer comprising about 1% to about 5% of at least one surface-active agent and about 1% to about 5% of at least one polymeric agent;

iii. water; and

iv. an active agent;

wherein the polymeric agent is about 0.01% to about 5% by weight and is selected from the group consisting of a bioadhesive agent, a gelling agent, a film forming agent and a phase change agent;

wherein the volatile glycerol ether evaporates upon application to a topical surface and is able to deposit a pharmaceutical or cosmetic agent for absorption onto the topical surface; and

wherein the composition is contained in a pressurized container and further comprises a liquefied hydrocarbon gas propellant at a concentration of about 3% to about 35% by weight of the total composition it is substantially flowable and provides a breakable thermolabile foam of about fairly good to about excellent quality upon release.

In one or more embodiments there is provided a pharmaceutical composition comprising:

i. a volatile glycerol ether selected from the group consisting of glycerol propyl ether, glycerol hexyl ether and glycerol nonyl ether and mixtures thereof;

ii. a stabilizer comprising about 1% to about 5% of at least one surface-active agent comprising a liquid surfactant and about 0.1% to about 5% of at least one polymeric agent;

iii. water; and

iv. an active agent

wherein the volatile glycerol ether is about 1% to about 85% by weight of the composition;

wherein the polymeric agent is about 0.01% to about 5% by weight and is selected from the group consisting of a bioadhesive agent, a gelling agent, a film forming agent and a phase change agent;

wherein the volatile glycerol ether evaporates upon application to a topical surface and is able to deposit a pharmaceutical or cosmetic agent for absorption onto the topical surface; and

wherein the composition is contained in a pressurized container and further comprises a liquefied hydrocarbon gas propellant at a concentration of about 3% to about 35% by weight of the total composition and is substantially flowable and provides a thermolabile foam of about fairly good to about excellent quality upon release.

In one or more embodiments there is provided a composition in a non-foam state.

In one or more embodiments there is provided a composition without propellant.

In one or more embodiments there is provided a vehicle or a therapeutic or a pharmaceutical composition in a non-foam state.

In one or more embodiments there is also provided a formulation of any of the compositions described herein for use in the manufacture of a medicament.

All % values are provided on a weight (w/w) basis.

### Glycerol Ethers

In an embodiment the organic carrier comprises one or more glycerol ethers which can be a volatile ether or a non-volatile ether or mixtures thereof. In a preferred embodiment the glycerol ethers are volatile ethers or a mixture of volatile and non-volatile ethers. The ethers themselves may comprise of a low or short side chain, a medium side chain, or a high or long side chain. They are often colorless, almost odorless liquids. For the purposes herein by volatile ethers it is intended liquid ethers that have a boiling point of about 70 degrees C. or less and similarly by non-volatile ethers it is intended ethers that have a boiling point of above about 70 degrees C. In some embodiments, volatile glycerol ethers useful in the instant compositions evaporate upon application to a surface at a temperature of about 37 degrees C.

Glycerol ethers have a number of interesting and unusual properties. They are amphiphilic and can constitute a new class of solvents. On the other hand they can have surfactant like properties (e.g. lowering interfacial tensions) and may form lamellar structures. As the alkyl chain is increased their hydrophobicity is increased. Branched alkyl ethers can prevent lamellar packing in water. Isostearyl glyceryl ether is an example of a branched chain alkyl glyceryl ether, which is said to have emulsion characteristics with water. They have been compared with polyethylene glycol type non-ionic surfactants. For example nonoxynol-9 is a PEG-9 nonyl phenyl ether, which is used as a surfactant and wetting agent. The phase behavior of alkyl glycerol ethers has been reported to be not dependant on temperature when compared to traditional non-ionic surfactants.

Glycerol ethers in general and volatile glycerol ethers in particular provide additional and unique complex challenges in producing compatible formulations that are able to carry and deliver active pharmaceutical ingredients to a target surface.

In an embodiment the ethers are glycerol ethers, derivatives and analogues thereof.

A general formula of glycerol ethers is ROCH₂CHOHCH₂OH. The glyceryol ethers may have an aliphatic hydrocarbon group having 4 to 12 carbon atoms, which may be a straight chain or, may have a branched chain, which may be an alkyl or an alkenyl group or an aromatic hydrocarbon group which may have a substituent. Non-limiting examples of the saturated straight-chain hydrocarbon groups include n-butyl, n-hexyl, n-octyl, n-nonyl, n-decyl, n-undecyl, n-dodecyl, and the like. Non-limiting examples of the branched hydrocarbon groups include iso-butyl, sec-butyl, tert-butyl, 3-methylbutyl, 4-methylpentyl, 5-methylhexyl, 2-ethylhexyl, 3,5,5-trimethylhexyl, 6-methylpentyl, 7-methyloctyl, 8-methylnonyl, 2,6-dimethylheptyl, 3,7-dimethyloctyl, and the like. Non-limiting examples of the unsaturated straight-chain hydrocarbon groups of include 1-butenyl, 2-butenyl, 3-butenyl, 4-pentenyl, 5-hexenyl, 6-heptenyl, 7-octenyl, 8-nonenyl, 9-decenyl, 1-pentenyl, 1-hexenyl-1-heptenyl, 1-octenyl, 1-nonenyl, 1-decenyl, 9-octadecenyl, and the like. Non-limiting examples of the-unsaturated hydrocarbon group which may have a branched chain of R include 2-methyl-1-propenyl, 3-methyl-2-butenyl, 4-methyl-3-pentenyl, 5-methyl-4-hexenyl, 6-methyl-5-heptenyl, 7-methyl-6-octenyl, 8-methyl-7-nonenyl, 1-methyl-1-propenyl, 1-methyl-1-butenyl, 1-methyl-1-pentenyl, 1-methyl-1-hexenyl, 1-methyl-1-heptenyl, 1-methyl-1-octenyl, 1-methyl-1-nonenyl, 2,6-dimethyl-5-heptenyl, 2,6-dimethyl-1-heptenyl, and the like. Non-limiting examples of the aromatic hydrocarbon group which may have a substituent include phenyl, 2-methylphenyl, 4-methylphenyl, 2,4,6-trimethylphenyl, 4-tert-butylphenyl, and the like. The number of groups is preferrably 1 or 2. Preferrably 2. Non-limiting examples are 3-methylbutoxypropane-1,2-diol, n-hexyloxypropane-1,2-diol, n-octyloxypropane-1,2-diol, 2-ethyl hexyloxypropane-1,2-diol, 3,5,5-trimethylhexyloxypropane-1,2-diol, n-decyloxypropane-1,2-diol, phenyloxypropane-1,2-diol, 2-methylphenyloxypropane-1,2-diol and 4-methylphenyloxypropane-1,2-diol,

Glycerol monoalkyl ethers have antimicrobial effects. Non-limiting examples of glycerol monoalkyl ethers include glycerol monoalkyl ethers substituted in the 1 or 2 position (i.e. symmetrical or asymmetrical) by saturated or unsaturated, branched or unbranched alkyl, such as dodecyl glycerol ether, decyl glycerol ether, nonyl glycerol ether, octyl glycerol ether, hexyl glycerol ether, propyl glycerol ether, octadecyl glycerol ether (batyl alcohol), hexadecyl glycerol ether (chimyl alcohol), menthyl glycerol ether and octadecenyl glycerol ether (selachyl alcohol),1-(2-ethylhexyl) glycerol ether and dodecyl glycerol ether.

Non-limiting examples of volatile ethers are (2E,4E,6E,12E,14E,16E)-2,4,6,12,14,16-octadecahexaene-9-yne-8,11-diol; 2,5,9,14,18,21-hexamethyl-4,6,8,10,12,14,16,18-docosaoctaene-2,21-diol; 2,6,10,14,19,23,27,31-octamethyl-2,6,8,10,12,14,16,18,20,22,24,26,30-dotriacontamidecaene-4,29-diol; propane-1,2,3-triol; 3-ethoxypropane-1,2-diol; 3-propoxypropane-1,2-diol; 3-butoxypropane-1,2-diol; 3-isobutoxypropane-1,2-diol; 3-tert-butoxypropane-1,2-diol; 3-(3-methylbutoxy)propane-1,2-diol; 3-(octyloxy)-1,2-propanediol; 3-(decyloxy)-1,2-propanediol; 3-(dodecyloxy)-1,2-propanediol; glycerol diethyl ether; glycerol vinyl ether; glycerol hexyl ether, glycerol nonyl ether; glycerol propyl ether; glycerol ethyl ether; glycerol methyl ether; glycerol butyl ether;

Other non-limiting examples of ethers are 2-Ethylhexyl glycerol ether, diethylene glycol monopentyl ether; 3-(tetradecyloxy)-1,2-propanediol; (S)-3-(hexadecyloxy)-1,2-propanediol; (R)-3-(hexadecyloxy)-1,2-propanediol, rac-(R̂*)-3-(hexadecyloxy)-1,2-propanediol; 2-(hexadecyloxy)propane-1,3-diol; (S)-3-(octadecyloxy)propane-1,2-diol; (R)-3-(octadecyloxy)propane-1,2-diol; 1-glycerol ether of 17Z-tetracosenol; glycerol isopropyl ether; glycerol isopentyl ether; diglycerol isopropyl ether; diglycerol isobutyl ether; triglycerol isopropyl ether;

Non-limiting examples of non-volatile ethers are Glycerol, 1(2-chlorophenyl)ether; glycerol, 1-(4-chlorophenyl)ether; glycerol, 1,2-dimethyl ether; glycerol, 1,3-dimethyl ether; glycerol, 1,3-diphenylether; glycerol, 1-(2-methoxyphenyl)ether; glycerol, 1-methyl ether; glycerol, 2-methyl ether; glycerol, 1-octadecyl ether; glycerol, 1-phenyl ether; and polyoxypropylene glyceryl ether.

Some ethers may have a pharmacological, a therapeutic, or a cosmetic effect. A couple of non-limiting examples are set out in the Table below.

| Item | Ether | Function |
|---|---|---|
| Guai- | Guaiacol Glycerol Ether | Expectorant |
| fenesin | (RS)-3-(2-Methoxyphenoxy)propane- | |
| | 1,2-diol | |
| Chlor- | p-Chlorophenyl α-Glyceryl Ether | Antifungal, antibac- |
| phenesyn | 3-(4-Chlorophenoxy)propane-1,2-diol | terial, and preservative |

In an embodiment, the ethers are selected from the group consisting of glycerol propyl ether, glycerol hexyl ether, glycerol nonyl ether, n butyl glycerol ether, diethyl glycerol monopentylether or mixtures thereof. In an embodiment, the ethers are a mixture of glycerol propyl ether, glycerol hexyl ether and glycerol nonyl ether.

It has been reported that as the alkyl chain length increases of alkyl (C₆, C₈, C₁₂ and iso-C₈) monoglycerol ethers and 2-hydroxyalkyl (C₆, C₈, C₁₂) mono glycerol ethers the tendency to form lamellar liquid crystalline phases increases. The branched alkyl chain in contrast is said to show no formation of lamellar phases even at high concentrations of surfactant. Hexyl glycerol ether is reported to have a c.m.c. of 15 mM and surface tension of 26 mN m⁻¹.

### Wax

In an embodiment, the organic carrier comprises a wax. In a preferred embodiment the wax is dissolvable or miscible in an ether. By wax is meant in the wider sense, waxes, waxy substances, counterparts and derivatives thereof. Waxes may be natural or artificial and include any organic material having wax-like properties.

A wax can be a solid wax or a liquid wax. For the purposes herein wax includes waxy substances, like fatty acids and their fatty alcohol counterparts, which can be short, medium and long chain. The fatty acid or alcohol backbone may be straight, branched, saturated, unsaturated, or hydrogenated, unhydrogenated, natural, or synthetic. Where one type of backbone produces a waxy substance then molecules with substantially the same backbone are also deemed as being part of the wax family or being a waxy substance counterpart or derivative thereof. For example, stearic acid, which is a waxy solid, has a C18 backbone. In other cases where the carbon backbone chain is C18, such as stearyl alcohol a solid and isostearic acid, oleic acid and oleyl alcohol, which are liquids, are all considered to be waxy substances, having a commonality with regards to the number of carbon atoms in the formula. Also within the scope is where hydrogenation would form a wax or waxy substance. In a preferred embodiment the wax is a liquid wax. Liquid waxes can in an embodiment help to thin or reduce the viscosity of the pre-foam formulations. They can also improve the sensory qualities and look and feel of the resultant foam. Non-limiting examples of liquid waxes are oleyl alcohol, isostearyl alcohol, capric alcohol, capryl alcohol, isostearic acid, caprylic acid, caproic acid, and butyric acid, and also jojoba oil.

Other non-limiting examples of fatty acids and fatty alcohols having a commonality of formula of backbone C chain are shown in Table 1:

TABLE 1

| Saturated | Solid = S | C Formula | Unsaturated | Solid = S | C Formula | Fatty | Solid = S | C Formula |
|---|---|---|---|---|---|---|---|---|
| Acid | Liquid = L | Backbone | acid | Liquid = L | Backbone | Alcohol | Liquid = L | Backbone |
| Butyric | L | C4:0 | | | | | | |
| Caproic | L | C6:0 | | | | | | |
| Caprylic | L | C8:0 | | | | capryl | L | C8:0 |
| Capric | S | C10:0 | | | | capric | L | C10:0 |
| Lauric | S | C12:0 | | | | 1-dodecanol | S | C12:0 |
| Myristic | S | C14:0 | Myristoleic acid: | S | C14:1 | Myristyl | S | C14:0 |
| Palmitic | S | C16:0 | | | | cetyl | S | C16:0 |
| | | | Palmitoleic acid: | L | C16:1 | palmitoleyl | | C16:1 |
| Stearic | S | C18:0 | Oleic acid: | L | C18:1 | stearyl | S | C18:0 |
| Isostearic | L | C18:B | | | | isostearyl | L | C18:B |
| | | | | | | oleyl | L | C18:1 |
| | | | Linoleic acid: | L | C18:2 | | | |
| | | | Alpha-linolenic acid: | L | C18:3 | | | |
| Arachidic | S | C20:0 | Arachidonic acid | L | C20:4 | arachidyl | S | C20:0 |
| | | | Eicosapentaenoic | S | C20:5 | | | |
| | | | acid | | | | | |
| Behenic | S | C22:0 | Erucic acid: | S | C22:1 | behenyl | S | C22:0 |
| | | | Docosahexaenoic | S | C22:6 | | | |
| | | | acid | | | | | |

Solid waxes that are suitable are those that are readily miscible or dissolvable in ethers. Waxes that are liquid at room temperature are potentially suitable as solvents and easier to use at higher concentrations.

In one or more embodiments the wax, waxy substance, counterpart or derivative thereof is a saturated branched fatty acid or fatty alcohol.

In one embodiment fatty acids which may be used in the fatty acid waxy substances, counterparts or derivatives include those having an alkyl or alkenyl group having 12 or more carbon atoms, preferably 14 to 22 carbon atoms. Examples of such fatty acid waxes are fatty acids such as lauric acid, myristic acid, palmitic acid, stearic acid, behenic acid, oleic acid, 12-hydroxystearic acid, undecylenic acid, tall acid, isostearic acid, linoleic acid, linolenic acid, eicosapentaenoic acid (EPA), docosahexaenoic acid (DHA), or the equivalent fatty alcohols.

In another embodiment composite fatty acids such as from coconut oil, palm oil, tallow and jojobo oil may be used.

In one or more embodiments, fatty acids can be solid substances and in one or more embodiments they may be liquid. In other embodiments a combination of liquid and solid substances is used.

Skin feeling can be improved when two or more fatty acids such as stearic acid and isostearic acid are used in combination. The same reasoning is applicable with appropriate changes to fatty alcohols.

In one or more embodiments, the fatty acids can act as one or more of a solvent, as a emollient, as a penetration enhancer. To the extent they are miscible with ethers they can be complimentary to the ether formulations.

In one or more embodiments, the fatty acids can act as a foam adjuvant.

In one or more embodiments, the fatty alcohol is oleyl alcohol. Oleyl alcohol is soluble in ethanol (95%), and ether but practically insoluble in water and should be stored in a well-closed container in a cool, dry, place. Oleyl alcohol and propylene glycol are miscible although they are hydrophobic and hydrophilic and therefore it is predicted that it should be possible to make substantially waterless or even waterless waxy or waxy substances foamable compositions and foam by reducing or replacing the water with an ether miscible polyol like propelene glycol. To the extent that isostearic acid and propylene gylycol or stearic acid and propylene glycol are likewise miscible the same will apply to them.

In one or more embodiments, the fatty acid is isostearic acid, oleic acid or stearic acid.

Oleic acid is miscible with ethanol (95%), ether, hexane, and fixed and volatile oils, but practically insoluble in water. Stearic acid (octadecanoic acid) soluble in ethanol (95%), hexane, and propylene glycol but is practically insoluble in water.

In one or more embodiments, isostearic acid and stearic acid are used in combination.

In one or more embodiments, oleyl alcohol and stearic acid are used in combination.

In one or more embodiments, oleyl alcohol and stearic acid are used in combination.

In one or more embodiments, at least any two from the group consisting of caprilic acid, capryl alcohol, capric acid, capric alcohol, palmitic acid, cetyl alcohol, stearic acid, isostearic acid, stearyl alcohol, and oleyl alcohol are used in combination.

Jojoba oil (pronounced “ho-HO-bah”) is a liquid straight chain wax ester, 36 to 46 carbon atoms in length that is chemically very similar to human sebum. Each molecule consists of a fatty acid and a fatty alcohol joined by an ester bond. Each molecule has two points of cis-unsaturation, both located at the 9th carbon atom from either end of the molecule. Jojoba oil comprises approximately 66-71% eicosenoic acid, 14-20% docosenoic acid and 10-13% oleic acid. Therapeutically it can aid in the healing process.

In one or more embodiments, the fatty acid or alcohol is a biologically active. For example, benhenyl alcohol has some antiviral properties apart from being a foam adjuvant or co-surfactant. In an embodiment, the biologically active fatty acid or alcohol possesses keratolytic activities.

In an embodiment, the waxy substance is incorporated in the foamable composition in a safe and effective amount. The term “safe and effective” means an amount of an active agent that exerts a therapeutic effect on a specific disorder, without causing adverse effects that may prohibit the use of said active agent in the treatment of said disorder.

In one or more embodiments, the wax, waxy substance, counterpart or derivative thereof contributes to the foam structure.

### Foam Adjuvant

Optionally, the foamable vehicle further includes a foam adjuvant selected from the group consisting of a fatty alcohol having 15 or more carbons in their carbon chain; a fatty acid having 16 or more carbons in their carbon chain; fatty alcohols, derived from beeswax and including a mixture of alcohols, a majority of which has at least 20 carbon atoms in their carbon chain; a fatty alcohol having at least one double bond; a fatty acid having at least one double bond; a branched fatty alcohol; a branched fatty acid and a fatty acid substituted with a hydroxyl group

In one or more embodiments the foam adjuvant is a wax, waxy substance, counterparts or derivative thereof.

### Additional Organic Carrier

Optionally, the foamable vehicle further includes at least one additional organic carrier selected from the group consisting of a hydrophobic organic carrier, an emollient and mixtures thereof, e.g. at a concentration of about 2% to about 50% by weight. The hydrophobic solvent and/or the emollient can be selected from the group consisting of mineral oil, triglycerides, capric/caprylic triglyceride, alkyl esters of fatty acids such as isopropyl palmitate, isopropyl isostearate, octyl palmitate, cetyl lactate, cetyl ricinoleate, tocopheryl acetate, acetylated lanolin alcohol, cetyl acetate, cetestearyl alcohol, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, maleated soybean oil, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxane, polyaryl siloxane, polyalkylaryl siloxane, a polyether siloxane copolymer and a poly(dimethylsiloxane)-(diphenyl-siloxane) copolymer.

In an embodiment, the organic carrier is a polypropylene glycol alkyl ether (PPG alkyl ether). PPG alkyl ethers are liquid, water-insoluble propoxylated fatty alcohols, having the molecular formula of RO(CH₂CHOCH₃)ₙ; wherein “R” is a straight-chained or branched C₄ to C₂₂ alkyl group; and “n” is in the range between 4 and about 50. PPG alkyl ethers are organic liquids that function as skin-conditioning agent in pharmaceutical and cosmetic formulations. Non-limiting exemplary PPG alkyl ethers include PPG stearyl ethers and PPG Butyl Ether. Preferred PPG alky ethers for use in the foamable compositions include PPG-15 Stearyl Ether, PPG-2 Butyl Ether, PPG-9-13 Butyl Ether and PPG-40 Butyl Ether.

In an embodiment, the organic carrier is a paraffin, glycerin or a petrolatum, which is also termed “white petrolatum” and “Vaseline”. Preferably they are used at between about 1% to about 5%.

In an embodiment, the organic carrier comprises a silicone, which can be volatile or non-volatile. Cyclomethicone is for example, a suitable volatile silicone. The non-volatile silicones are preferably liquid. The use of silicones can compliment and support the use of volatile and non-volatile ethers.

In an embodiment, the organic carrier comprises a polyol liquid, and/or a polar solvent and/or a short chain alcohol, and/or a glycol, and/or a non-polar carrier.

### Polymeric Agent

The composition contains a polymeric agent selected from the group consisting of a bioadhesive agent, a gelling agent, a film forming agent and a phase change agent. A polymeric agent enhances the creation of foam having fine bubble structure, which does not readily collapse upon release from the pressurized aerosol can. The polymeric agent serves to stabilize the foam composition and to control drug residence in the target organ.

Exemplary polymeric agents include, in a non-limiting manner, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, amine-bearing polymers such as chitosan; acidic polymers obtainable from natural sources, such as alginic acid and hyaluronic acid; chemically modified starches and the like, carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like.

Additional exemplary polymeric agents include semi-synthetic polymeric materials such as cellulose ethers, such as methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, and cationic celluloses, carbomer (homopolymer of acrylic acid is crosslinked with an allyl ether pentaerythritol, an allyl ether of sucrose, or an allyl ether of propylene, such as Carbopol® 934, Carbopol® 940, Carbopol® 941, Carbopol® 980 and Carbopol® 981, pemulen, klucel, and aluminum starch octenylsuccinate (ASOS) or other derivatized polymers. Polyethylene glycol, having molecular weight of 1000 or more (e.g., PEG 1,000, PEG 4,000, PEG 6,000 and PEG 10,000) also have gelling capacity and while they are considered herein as “secondary polar solvents”, as detailed herein, they are also considered polymeric agents.

In one or more embodiments the polymeric agents have emulsifying properties. In certain preferred embodiments the polymeric agent is a derivatized hydrophilic polymer with hydrophobic alkyl moieties Other types that may also a similar stabilizing effect are silicone copolymers and derivatized starch ASOS.

Mixtures of the above polymeric agents are contemplated.

The concentration of the polymeric agent should be selected so that the composition, after filling into aerosol canisters, is flowable, and can be shaken in the canister. In one or more embodiments, the concentration of the polymeric agent is selected such that the viscosity of the composition, prior to filling of the composition into aerosol canisters, is about or less than 12,000 CPs, and more preferably, less than 10,000 CPs. Nevertheless where waxy substances are used in the composition especially in higher concentrations the viscosity may substantially exceed these figures.

In one or more embodiments the polymeric agent is a water miscible polymeric agent.

In one or more embodiments the polymeric agent is selected from at least one of the group consisting of avicel, pemulen, carboxymethyl cellulose (CMC), carbomer, klucel, hydroxyl propyl methyl cellulose and xantham gum.

Avicel comprises microcrystalline cellulose which in water, with shear, forms a matrix comprised of insoluble microcrystals that can result in a stable and thixotropic formulation. In a preferred embodiment, the polymer comprises microcrystalline cellulose in combination with another polymer such as CMC.

### Surface Active Agent

The composition further contains a surface-active agent. Surface-active agents (also termed “surfactants”) include any agent linking oil and water in the composition, in the form of emulsion. A surfactant's hydrophilic/lipophilic balance (HLB) describes the emulsifier's affinity toward water or oil. HLB is defined for non-ionic surfactants. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average). In many cases a single surfactant may suffice. In other cases a combination of two or more surfactants is desired. Reference to a surfactant in the specification can also apply to a combination of surfactants or a surfactant system. As will be appreciated by a person skilled in the art, which surfactant or surfactant system is more appropriate is related to the vehicle and intended purpose. In general terms a combination of surfactants is usually preferable where the vehicle is an emulsion. In an emulsion environment a combination of surfactants can be significant in producing breakable foams of good quality. It has been further discovered that the generally thought considerations for HLB values for selecting a surfactant or surfactant combination are not always binding for emulsions and that good quality foams can be produced with a surfactant or surfactant combination both where the HLB values are in or towards the lipophilic side of the scale and where the HLB values are in or towards the hydrophilic side of the scale. Surfactants also play a role in foam formation where the foamable formulation is a single phase composition.

According to one or more embodiments the composition contains a single surface active agent having an HLB value between about 2 and 9, or more than one surface active agent and the weighted average of their HLB values is between about 2 and about 9. Lower HLB values may in certain embodiments be more applicable to water in oil emulsions.

According to one or more embodiments the composition contains a single surface active agent having an HLB value between about 7 and 14, or more than one surface active agent and the weighted average of their HLB values is between about 7 and about 14. Mid range HLB values may in certain embodiments be more suitable for oil in water emulsions.

According to one or more other embodiments the composition contains a single surface active agent having an HLB value between about 9 and about 19, or more than one surface active agent and the weighted average of their HLB values is between about 9 and about 19. In a waterless or substantially waterless environment a wide range of HLB values may be suitable.

Preferably, the composition contains a non-ionic surfactant. Non-limiting examples of possible non-ionic surfactants include a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkyl ether, polyoxyethylene cetyl ether, polyoxyethylene palmityl ether, polyethylene oxide hexadecyl ether, polyethylene glycol cetyl ether, steareths such as steareth 2, brij 21, brij 721, brij 38, brij 52, brij 56 and brij W1, a sucrose ester, a partial ester of sorbitol and its anhydrides, sorbitan monolaurate, sorbitan monolaurate, a monoglyceride, a diglyceride, isoceteth-20 and mono-, di- and tri-esters of sucrose with fatty acids. In certain embodiments, suitable sucrose esters include those having high monoester content, which have higher HLB values.

In an embodiment the surfactant is an ether for example polyoxyethylene (26) glycerol ether.

In certain embodiments, surfactants are selected which can provide a close packed surfactant layer separating the oil and water phases. To achieve such objectives combinations of at least two surfactants are selected. Preferably, they should be complex emulgators and more preferably they should both be of a similar molecular type; for example, a pair of ethers, like steareth 2 and steareth 21, or a pair of esters, for example, PEG-40 stearate and polysorbate 80. Ideally, the surfactants can be ethers. In certain circumstances POE esters cannot be used and a combination of sorbitan laurate and sorbitan stearate or a combination of sucrose stearic acid ester mixtures and sodium laurate may be used. All these combinations due to their versatility and strength may also be used satisfactorily and effectively with ether formulations, although the amounts and proportion may be varied according to the formulation and its objectives as will be appreciated by one of skill in the art.

It has been discovered also that by using a derivatized hydrophilic polymer with hydrophobic alkyl moieties as a polymeric emulsifier such as pemulen it is possible to stabilize the emulsion better about or at the region of phase reversal tension. Other types of derivatized polymers like silicone copolymers, derivatized starch [Aluminum Starch Octenylsuccinate (ASOS)]/[DRY-FLO AF Starch], and derivatized dexrin may also have a similar stabilizing effect.

A series of dextrin derivative surfactants prepared by the reaction of the propylene glycol polyglucosides with a hydrophobic oxirane-containing material of the glycidyl ether are highly biodegradable. [Hong-Rong Wang and Keng-Ming Chen, Colloids and Surfaces A: Physicochemical and Engineering Aspects Volume 281, Issues 1-3, 15 Jun. 2006, Pages 190-193].

Non-limiting examples of non-ionic surfactants that have HLB of about 7 to about 12 include steareth 2 (HLB˜4.9); glyceryl monostearate/PEG 100 stearate (Av HLB˜11.2); stearate Laureth 4 (HLB˜9.7) and cetomacrogol ether (e.g., polyethylene glycol 1000 monocetyl ether).

Non-limiting examples of preferred surfactants, which have a HLB of 4-19 are set out in the Table below:

| Surfactant | HLB |
|---|---|
| steareth 2 | ~4.9 |
| glyceryl monostearate/PEG 100 stearate | Av ~11.2 |
| Glyceryl Stearate | ~4 |
| Steareth-21 | ~15.5 |
| peg 40 stearate | ~16.9 |
| polysorbate 80 | ~15 |
| sorbitan stearate | ~4.7 |
| laureth 4 | ~9.7 |
| Sorbitan monooleate (span 80) | ~4.3 |
| ceteareth 20 | ~15.7 |
| steareth 20 | ~15.3 |
| ceteth 20 | ~15.7 |
| Macrogol Cetostearyl Ether | ~15.7 |
| ceteth 2 (Lipocol C-2) | ~5.3 |
| PEG-30 Dipolyhydroxystearate | ~5.5 |
| sucrose distearate (Sisterna SP30) | ~6 |
| polyoxyethylene (100) stearate | ~18.8 |

More exemplary stabilizing surfactants which may be suitable for use in the present foamable compositions are found below.

PEG-Fatty Acid Monoester Surfactants

| Chemical name | Product example name | HLB |
|---|---|---|
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-45 stearate | Nikkol MYS-45 (Nikko) | 18 |
| PEG-50 stearate | Myrj 53 | >10 |
| PEG-100 stearate | Myrj 59, Arlacel 165 (ICI) | 19 |

PEG-Fatty Acid Diester Surfactants:

| Chemical name | Product example name | HLB |
|---|---|---|
| PEG-4 dilaurate | Mapeg ™ 200 DL (PPG), | 7 |
| | Kessco ™ PEG 200 DL | |
| | (Stepan), LIPOPEG 2-DL (Lipo | |
| | Chem.) | |
| PEG-4 | distearate Kessco ™ 200 DS | 5 |
| | (Stepan.sub) | |
| PEG-32 dioleate | Kessco ™ PEG 1540 DO | 15 |
| | (Stepan) | |
| PEG-400 dioleate | Cithrol 4DO series (Croda) | >10 |
| PEG-400 disterate | Cithrol 4DS series (Croda) | >10 |
| PEG-20 glyceryl oleate | Tagat ™ O (Goldschmidt) | >10 |

Transesterification Products of Oils and Alcohols

| Chemical name | Product example name | HLB |
|---|---|---|
| PEG-30 castor oil | Emalex C-30 (Nihon Emulsion) | 11 |
| PEG-40 hydrogenated | Cremophor RH 40 (BASF), | 13 |
| castor oil | Croduret (Croda), Emulgin | |
| | HRE 40 (Henkel) | |

Polyglycerized Fatty Acids, such as:

| Chemical name | Product example name | LB |
|---|---|---|
| Polyglyceryl-6 dioleate | Caprol ™ 6G20 (ABITEC); | 8.5 |
| | PGO-62 (Calgene), PLUROL | |
| | OLEIQUE CC 497 (Gattefosse) | |
| | Hodag | |

PEG-Sorbitan Fatty Acid Esters

| Chemical name | Product example name | HLB |
|---|---|---|
| PEG-20 sorbitan | Tween-20 (Atlas/ICI), Crillet 1 | 17 |
| monolaurate | (Croda), DACOL MLS 20 | |
| | (Condea) | |
| PEG-20 sorbitan | Tween 40 (Atlas/ICI), Crillet 2 | 16 |
| Monopalmitate | (Croda) | |
| PEG-20 sorbitan | Tween-60 (Atlas/ICI), Crillet 3 | 15 |
| monostearate | (Croda) | |
| PEG-20 sorbitan | Tween-80 (Atlas/ICI), Crillet 4 | 15 |
| monooleate | (Croda) | |

Polyethylene Glycol Alkyl Ethers

| Chemical name | Product example name | HLB |
|---|---|---|
| PEG-2 oleyl ether | oleth-2 Brij 92/93 (Atlas/ICI) | 4.9 |
| PEG-3 oleyl ether | oleth-3 Volpo 3 (Croda) | <10 |
| PEG-5 oleyl ether | oleth-5 Volpo 5 (Croda) | <10 |
| PEG-10 oleyl ether | oleth-10 Volpo 10 (Croda), Brij | 12 |
| | 96/97 (Atlas/ICI) | |
| PEG-20 oleyl ether | oleth-20 Volpo 20 (Croda), Brij | 15 |
| | 98/99 (Atlas/ICI) | |
| PEG-4 lauryl ether | laureth-4Brij 30 (Atlas/ICI) | 9.7 |
| PEG-23 lauryl ether | laureth-23Brij 35 (Atlas/ICI) | 17 |
| PEG-10 stearyl ether | Brij 76 (ICI) | 12 |
| PEG-2 cetyl ether | Brij 52 (ICI) | 5.3 |

Sugar Ester Surfactants

| Chemical name | Product example name | HLB |
|---|---|---|
| Sucrose distearate | Sisterna SP50, Surfope 1811 | 11 |

Sorbitan Fatty Acid Ester Surfactants

| Chemical name | Product example name | HLB |
|---|---|---|
| Sorbitan monolaurate | Span-20 (Atlas/ICI), Crill 1 | 8.6 |
| | (Croda), Arlacel 20 (ICI) | |
| Sorbitan monopalmitate | Span-40 (Atlas/ICI), Crill 2 | 6.7 |
| | (Croda), Nikkol SP-10 (Nikko) | |
| Sorbitan monooleate | Span-80 (Atlas/ICI), Crill 4 | 4.3 |
| | (Croda), Crill 50 (Croda) | |
| Sorbitan monostearate | Span-60 (Atlas/ICI), Crill 3 | 4.7 |
| | (Croda), Nikkol SS-10 (Nikko) | |

In one or more embodiments the surface active agent is a complex emulgator in which the combination of two or more surface active agents can be more effective than a single surfactant and provides a more stable emulsion or improved foam quality than a single surfactant. For example and by way of non-limiting explanation it has been found that by choosing e.g. two surfactants, one hydrophobic and the other hydrophilic, the combination can produce a more stable emulsion than a single surfactant. Preferably, the complex emulgator comprises a combination of surfactants wherein there is a difference of about 4 or more units between the HLB values of the two surfactants or there is a significant difference in the chemical nature or structure of the two or more surfactants.

Specific non-limiting examples of surfactant systems are, combinations of polyoxyethylene alkyl ethers, such as Brij 59/Brij 10; Brij 52/Brij 10; Steareth 2/Steareth 20; Steareth 2/Steareth 21 (Brij 72/Brij 721); combinations of polyoxyethylene stearates such as Myrj 52/Myrj 59; combinations of sucrose esters, such as Surphope 1816/Surphope 1807; combinations of sorbitan esters, such as Span 20/Span 80; Span 20/Span 60; combinations of sucrose esters and sorbitan esters, such as Surphope 1811 and Span 60; combinations of liquid polysorbate detergents and PEG compounds, such as Tween 80/PEG-40 stearate; methyl glucaso sequistearate; polymeric emulsifiers, such as Permulen (TR1 or TR2); liquid crystal systems, such as Arlatone (2121), Stepan (Mild RM1), Nikomulese (41) and Montanov (68) and the like.

In certain embodiments the surfactant is preferably one or more of the following: a combination of steareth-2 and steareth-21 on their own or in combination with glyceryl monostearate (GMS); in certain other embodiments the surfactant is a combination of polysorbate 80 and PEG-40 stearate. In certain other embodiments the surfactant is a combination of glyceryl monostearate/PEG 100 stearate. In certain other embodiments the surfactant is a combination of two or more of stearate 21, PEG 40 stearate, and polysorbate 80. In certain other embodiments the surfactant is a combination of two or more of laureth 4, span80, and polysorbate 80. In certain other embodiments the surfactant is a combination of two or more of GMS and ceteareth. In certain other embodiments the surfactant is a combination of two or more of steareth 21, ceteareth 20, ceteth 2 and laureth 4. In certain other embodiments the surfactant is a combination of ceteareth 20 and polysorbate 40 stearate. In certain other embodiments the surfactant is a combination of span 60 and GMS. In certain other embodiments the surfactant is a combination of two or all of PEG 40 stearate, sorbitan stearate and polysorbate 60

In certain other embodiments the surfactant is one or more of sucrose stearic acid esters, sorbitan laureth, and sorbitan stearate.

Without being bound by any particular theory or mode of operation, it is believed that the use of non-ionic surfactants with significant hydrophobic and hydrophilic components, increase the emulsifier or foam stabilization characteristics of the composition. Similarly, without being bound by any particular theory or mode of operation, using combinations of surfactants with high and low HLB's to provide a relatively close packed surfactant layer may strengthen the emulsion.

In one or more embodiments the stability of the composition can be improved when a combination of at least one non-ionic surfactant having HLB of less than 9 and at least one non-ionic surfactant having HLB of equal or more than 9 is employed. The ratio between the at least one non-ionic surfactant having HLB of less than 9 and the at least one non-ionic surfactant having HLB of equal or more than 9, is between 1:8 and 8:1, or at a ratio of 4:1 to 1:4. The resultant HLB of such a blend of at least two emulsifiers is preferably between about 9 and about 14.

Thus, in an exemplary embodiment, a combination of at least one non-ionic surfactant having HLB of less than 9 and at least one non-ionic surfactant having HLB of equal or more than 9 is employed, at a ratio of between 1:8 and 8:1, or at a ratio of 4:1 to 1:4, wherein the HLB of the combination of emulsifiers is preferably between about 5 and about 18.

In certain cases, the surface active agent is selected from the group of cationic, zwitterionic, amphoteric and ampholytic surfactants, such as sodium methyl cocoyl taurate, sodium methyl oleoyl taurate, sodium lauryl sulfate, triethanolamine lauryl sulfate and betaines.

Many amphiphilic molecules can show lyotropic liquid-crystalline phase sequences depending on the volume balances between the hydrophilic part and hydrophobic part. These structures are formed through the micro-phase segregation of two incompatible components on a nanometer scale. Soap is an everyday example of a lyotropic liquid crystal. Certain types of surfactants tend to form lyotropic liquid crystals in emulsions interface (oil-in-water) and exert a stabilizing effect.

In one or more embodiments the surfactant is a surfactant or surfactant combination which is capable of or which tends to form liquid crystals. Surfactants which tend to form liquid crystals may improve the quality of foams. Non-limiting examples of surfactants with postulated tendency to form interfacial liquid crystals are: phospholipids, alkyl glucosides, sucrose esters, sorbitan esters.

In one or more embodiments the at least one surface active agent is liquid. Moreover for the purposes of formulating with liquid ethers a liquid surfactant is preferred.

In one or more embodiments the liquid surfactant is a polysorbate, preferably polysorbate 80 or 60.

In one or more embodiments the at least one surface active agent is solid, semi solid or waxy.

It should be noted that HLB values may not be so applicable to non-ionic surfactants, for example, with liquid crystals or with silicones. Also HLB values may be of lesser significance in a waterless or substantially non-aqueous environment.

In one or more embodiments the surfactant can be, a surfactant system comprising a surfactant and a co-surfactant, a waxy emulsifier, a liquid crystal emulsifier, an emulsifier which is solid or semi-solid at room temperature and pressure, or combinations of two or more agents in an appropriate proportion as will be appreciated a person skilled in the art. Where a solid or semi-solid emulsifier combination is used it can also comprise a solid or semi-solid emulsifier and a liquid emulsifier. In one embodiment at least one surfactant is a liquid.

In one or more embodiments, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. Non-ionic surfactants alone can provide formulations and foams of good or excellent quality in the carriers and compositions herein.

Thus, in one embodiment, the composition contains a non-ionic surfactant as the surface active agent. In another preferred embodiment the composition includes a mixture of non-ionic surfactants as the sole surface active agent. Yet, in additional embodiments, the foamable composition includes a mixture of at least one non-ionic surfactant and at least one ionic surfactant in a ratio in the range of about 100:1 to 6:1. In one or more embodiments, the non-ionic to ionic surfactant ratio is greater than about 6:1, or greater than about 8:1; or greater than about 14:1, or greater than about 16:1, or greater than about 20:1. In further embodiments, the surface active agent comprises a combination of a non-ionic surfactant and an ionic surfactant, at a ratio of between 1:1 and 20:1

In one or more embodiments, a combination of a non-ionic surfactant and an ionic surfactant (such as sodium lauryl sulphate and cocamidopropylbetaine) is employed, at a ratio of between 1:1 and 20:1, or at a ratio of 4:1 to 10:1; for example, about 1:1, about 4:1, about 8:1, about 12:1, about 16:1 and about 20:1 or at a ratio of 4:1 to 10:1, for example, about 4:1, about 6:1, about 8:1 and about 10:1.

In selecting a suitable surfactant or combination thereof it should be borne in mind that the upper amount of surfactant that may be used may be limited by the shakability of the composition. If the surfactant is non-liquid, it can make the formulation viscous or solid. Subject to its miscibily solid surfactants should be first added to the aqueous phase, and may require gentle warming and then cooling before being combined with the ether phase. In general terms, as the amount of non-liquid surfactant is increased the shakability of the formulation reduces until a limitation point is reached where the formulation becomes non-shakable and unsuitable. Thus in one embodiment, an effective amount of surfactant may be used provided the formulation remains shakable. In other certain exceptional embodiments the upper limit may be determined by flowability such as in circumstances where the composition is marginally or apparently non-shakable. The formulation is sufficiently flowable to be able to flow through an actuator valve and be released and still expand to form a good quality foam.

In certain embodiments the amount of surfactant or combination of surfactants is between about 0.05% to about 20%; between about 0.05% to about 15%. or between about 0.05% to about 10%. In a preferred embodiment the concentration of surface active agent is between about 0.2% and about 8%. In a more preferred embodiment the concentration of surface active agent is between about 1% and about 6%.

In some embodiments, it is desirable that the surface active agent does not contain a polyoxyethylene (POE) moiety, such as polysorbate surfactants, POE fatty acid esters, and POE alkyl ethers, because the active agent is incompatible with such surface active agents. For example, the active agent pimecrolimus is not stable the presence of POE moieties, yet benefits greatly from the use of dicarboxylic esters as penetration enhancers. In such cases, alternative surface active agents are employed. In an exemplary manner, POE-free surfactants include non-ethoxylated sorbitan esters, such as sorbitan monopalmitate, sorbitan monostearate, sorbitan tristearate, sorbitan monooleate, sorbitan trioleate, sorbitan monolaurate and sorbitan sesquioleate; glycerol fatty acid esters, such as glycerol monostearate and glycerol monooleate; mono-, di- and tri-esters of sucrose with fatty acids (sucrose esters), sucrose stearate, sucrose distearate sucrose palmitate and sucrose laurate; and alkyl polyglycosides, such as lauryl diglucoside.

If the composition as formulated is a substantially non-shakable composition it is nevertheless possible as an exception in the scope for the formulation to be flowable to a sufficient degree to be able to flow through an actuator valve and be released and still expand to form a good quality foam. This surprising and unusual exception may be due one or more of a number of factors such as the high viscosity, the softness, the lack of crystals, the pseudoplastic or semi pseudo plastic nature of the composition and the dissolution of the propellant into the composition.

In one or more embodiments, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content, which have higher HLB values.

### Substantially Alcohol-Free

According to one or more embodiments, the foamable composition is substantially alcohol-free, i.e., free of short chain alcohols. Short chain alcohols, having up to 5 carbon atoms in their carbon chain skeleton and one hydroxyl group, such as ethanol, propanol, isopropanol, butaneol, iso-butaneol, t-butaneol and pentanol, are considered less desirable solvents or polar solvents due to their skin-irritating effect. Thus, the composition is substantially alcohol-free and includes less than about 5% final concentration of lower alcohols, preferably less than about 2%, more preferably less than about 1%.

### Substantially Non-Aqueous

In certain cases, the active agent degrades in the presence of water, and therefore, in such cases the present of water in the composition is not desirable. Thus, in certain preferred embodiments, the composition is substantially non-aqueous. The term “substantially non-aqueous” or “substantially waterless” is intended to indicate that the composition has a water content below about 25%.

### Shakability

‘Shakability’ means that the composition contains some or sufficient flow to allow the composition to be mixed or remixed on shaking. That is, it has fluid or semi fluid properties. In some very limited cases, possibly aided by the presence of silicone, it may exceptionally be possible to have a foamable composition which is flowable but not apparently shakable.

### Breakability

A breakable foam is one that is thermally stable, yet breaks under sheer force. The foam containing volatile ethers is easily breakable upon mechanical force yet is thermolabile with the volatile ethers evaporating upon exposure to body temperature environment. Easy breakablility with gentle or moderate force enables the foam to be spread with ease on a topical body surface and where appropriate cover large areas. It is particularly advantageous where the body surface includes sensitive or painful areas helping to minimize discomfort. As the ethers evaporate the foam reduces in size until it disappears leaving the nonvolatile contents deposited and/or absorbed on a topical surface. By altering the ether(s) used the rate of evaporation may be adjusted to facilitate comfortable application and well directed administration to the target area.

### Substantially Homogenous Distribution

In some embodiments, the vehicle composition contains a substantially homogenous distribution of a pharmaceutical or cosmetic agent. In other embodiments, the pharmaceutical or cosmetic agent can be substantially homogenously distributed in the vehicle composition. In other embodiments, the vehicle composition is able to carry a substantially homogenous distribution of a pharmaceutical or cosmetic agent, e.g., capable of forming a substantially homogenous mixture with a pharmaceutical or cosmetic agent.

The homogeneity of a composition containing a pharmaceutical or cosmetic agent and the vehicle composition can be evaluated qualitatively or quantitatively. For example, a pharmaceutical or cosmetic agent is substantially homogenously distributed in the vehicle composition when the agent is distributed substantially evenly or uniformly throughout the vehicle composition, e.g., such that the agent is not preferentially located in a particular phase of the composition or that the agent is not sequestered, thereby creating pockets of high concentration of agent in the vehicle composition. Alternatively, a pharmaceutical or cosmetic agent is substantially homogenously distributed in the vehicle composition if the concentration (w/w %) of the pharmaceutical or cosmetic agent in a 25 mL aliquot of the foamed vehicle composition is within 20% of the concentration (w/w %) of the pharmaceutical or cosmetic agent in the total volume of the foamed vehicle composition. Thus whether a distribution of a pharmaceutical or cosmetic agent is substantially homogenous is determined based on the vehicle composition in its foamed state, using the w/w % of the pharmaceutical or cosmetic agent in the foamed vehicle composition.

### Modulating Agent

The term modulating agent is used to describe an agent which can improve the stability of or stabilize a foamable carrier or composition and/or an active agent by modulating the effect of a substance or residue present in the carrier or composition.

In one or more embodiments the modulating agent is used in a single phase system. In one or more embodiments the modulating agent is used in an aqueous system. In one or more embodiments the modulating agent is used in a substantially non-aqueous system. In an embodiment it is used in an emulsion system. In one aspect, in a water in oil emulsion and in another aspect in an oil in water emulsion.

In certain embodiments the substance or residue may for example be acidic or basic and potentially alter pH or it may be one or more metal ions which may act as a potential catalyst.

In one or more embodiments the modulating agent is used to describe an agent which can affect pH in an aqueous solution. In one or more embodiments the modulating agent is used to describe an agent which can affect pH in a substantially non-aqueous solution. The agent can be any of the known buffering systems used in pharmaceutical or cosmetic formulations as would be appreciated by one of skill in the art. It can also be an organic acid, a carboxylic acid, a fatty acid an amino acid, an aromatic acid, an alpha or beta hydroxyl acid an organic base or a nitrogen containing compound.

In one or more further embodiments the modulating agent is used to describe an agent, which is a chelating or sequestering or complexing agent that is sufficiently soluble or functional in the solvent to enable it to “mop up” or “lock” metal ions.

In an embodiment the term modulating agent more particularly means an acid or base or buffer system or combinations thereof, which is introduced into or is present in and acts to modulate the ionic or polar characteristics and any acidity or basesity balance of an emulsion carrier, composition, foamable carrier or foamable composition or resultant foam.

In one or more embodiments the modulating agent comprises an organic compound.

In one or more preferred embodiments the chelating agent is selected from the group consisting of ethylenediaminetetraacetic acid (EDTA), diethylenetriaminepentaacetic acid (DTPA), hydroxyethylenediaminetriacetic acid (HEDTA), nitrilotriacetic acid (NTA), O,O′-bis(2-aminoethyl)ethyleneglycol-N,N,N′,N′-tetraacetic acid (EGTA), trans-1,2-diaminocyclohexane-N,N,N′,N′-tetraacetic acid (CYDTA) or a pharmaceutically acceptable salt thereof (normally as a sodium salt), more preferably EDTA, HEDTA and their salts; most preferably EDTA and its salts.

In one or more embodiments a preferred non-limiting example of the chelating agent is EDTA. Typically, the chelating and sequestering agent is present in the composition at a level of up to about 5.0%, preferably 1.0 percent, by weight, of the composition.

In one or more embodiments the modulating agent may also be a preservative or an antioxidant or an ionization agent. Any preservative, antioxidant or ionization agents suitable for pharmaceutical or cosmetic application may be used. Non-limiting examples of antioxidants are tocopherol succinate, propyl galate, butylated hydroxy toluene and butyl hydroxy anisol and flavanoids. Ionization agents may be positive or may be negative depending on the environment and the active agent or composition that is to be protected. Ionization agents may for example act to protect or reduce sensitivity of active agents. Non-limiting examples of positive ionization agents are benzyl conium chloride, and cetyl pyridium chloride. Non-limiting examples of negative ionization agents are sodium lauryl sulphate, sodium lauryl lactylate and phospholipids.

In one or more embodiments the modulating agent is a liquid modulating agent.

In an embodiment an effective amount of an antioxidant such as a tocopherol is used as a modulating agent for glycerol ether compositions. It may be, without being bound by any theory, that the antioxidant restricts or prevents the formation of peroxides.

In an embodiment a combination of several agents are used in the same formulation as the modulating agent. A non-limiting example is where there is present two or more of the following: a pH agent, a buffer, a chelating agent, an antioxidant and an ionization agent.

### Humectant

A humectant is a substance that helps retain moisture and also prevents rapid evaporation. Non-limiting examples are propylene glycol, propylene glycol derivatives, glycerin, hydrogenated starch hydrosylate, hydrogenated lanolin, lanolin wax, D manitol, sorbitol, sodium 2-pyrrolidone-5-carboxylate, sodium lactate, sodium PCA, soluble collagen, dibutyl phthalate, and gelatin. Other examples may be found in the Handbook of Pharmaceutical Additives published by Gower.

### Moisturizers

A moisturizer is a substance that helps retain moisture or add back moisture to the skin. Examples are allantoin, petrolatum, urea, lactic acid, sodium PCV, glycerin, shea butter, caprylic/capric/stearic triglyceride, candelilla wax, propylene glycol, lanolin, hydrogenated oils, squalene, sodium hyaluronate and lysine PCA. Other examples may be found in the Handbook of Pharmaceutical Additives published by Gower.

Pharmaceutical compositions may in one or more embodiments usefully comprise in addition a heumectant or a moisturizer or combinations thereof.

### Polar Solvent

Optionally, the foamable vehicle further includes at least one polar solvent.

A “polar solvent” is an organic solvent, typically soluble in both water and oil. Certain polar solvents, for example propylene glycol and glycerin, possess the beneficial property of a humectant.

In one or more embodiments, the polar solvent is a humectant.

In one or more embodiments, the polar solvent is a polyol. Polyols are organic substances that contain at least two hydroxy groups in their molecular structure. Polyols are a preferred polar substance for use with volatile ethers.

In one or more embodiments, the polar solvent contains an diol (a compound that contains two hydroxy groups in its molecular structure), such as propylene glycol (e.g., 1,2-propylene glycol and 1,3-propylene glycol), butaneediol (e.g., 1,4-butaneediol), butaneediol (e.g., 1,3-butaneediol and 1,4-butenediol), butynediol, pentanediol (e.g., 1,5-pentanediol), hexanediol (e.g., 1,6-hexanediol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

In one or more embodiments, the polar solvent contains a triol (a compound that contains three hydroxy groups in its molecular structure), such as glycerin and 1,2,6-Hexanetriol.

Other non-limiting examples of polar solvents include pyrrolidones, (such as N-methyl-2-pyrrolidone and 1-methyl-2-pyrrolidinone), dimethyl isosorbide, 1,2,6-hexapetriol, dimethyl sulfoxide (DMSO), ethyl proxitol, dimethylacetamide (DMAc) and alpha hydroxy acids, such as lactic acid and glycolic acid.

According to still other embodiments, the polar solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

Polar solvents are known to enhance the penetration of active agent into the skin and through the skin, and therefore, their inclusion in the composition can be desirable, despite their undesirable skin drying and irritation potential. There is at one level a commonality between the different polar solvents and their penetration enhancement properties. Lower molecular weight alcohols can sometimes be more potent as a solvent, for example by extracting lipids from the skin layers more effectively, which characteristic can adversely affect the skin structure and cause dryness and irritation. Therefore the selection of lower molecular weight alcohols is ideally avoided.

Polar solvents, such as detailed below, possess high solubilizing capacity and contribute to the skin penetration of an active agent. Non-limiting examples include dimethyl isosorbide polyols, such as glycerol (glycerin), propylene glycol, hexylene glycol, diethylene glycol, propylene glycol n-alkanols, terpenes, di-terpenes, tri-terpenes, limonene, terpene-ol, 1-menthol, dioxolane, ethylene glycol, other glycols, oleyl alcohol, alpha-hydroxy acids, such as lactic acid and glycolic acid, sulfoxides, such as dimethylsulfoxide (DMSO), dimethylformanide, methyl dodecyl sulfoxide, dimethylacetamide, azone (1-dodecylazacycloheptan-2-one), 2-(n-nonyl)-1,3-dioxolane, alkanols, such as dialkylamino acetates, and admixtures thereof. In certain preferred embodiments, the polar solvent is selected from the group consisting of dimethyl isosorbide glycerol (glycerin), propylene glycol, hexylene glycol, terpene-ol, oleyl alcohol, lactic acid and glycolic acid.

### Skin Penetration Enhancer

A “skin penetration enhancer”, also termed herein “penetration enhancer,” is an organic solvent, typically soluble in both water and oil. Examples of penetration enhancer include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, l-menthol, dioxolane, ethylene glycol, hexylene glycol, other glycols, sulfoxides, such as dimethylsulfoxide (DMSO), dimethylformanide, methyl dodecyl sulfoxide, dimethylacetamide, dimethylisosorbide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the penetration enhancer is a polyethylene glycol (PEG) or PEG derivative that is liquid at ambient temperature

### Potent Solvent

In one or more embodiments, the foamable composition includes a potent solvent, in addition to or in place of one of the hydrophobic solvents, polar solvents or emollients of the composition. A potent solvent is a solvent other than mineral oil that solubilizes a specific active agent substantially better than a hydrocarbon solvent such as mineral oil or petrolatum. For example, a potent solvent solubilizes the active agent 5-fold better than a hydrocarbon solvent; or even solubilizes the active agent 10-fold better than a hydrocarbon solvent.

In one or more embodiments, the composition includes at least one active agent in a therapeutically effective concentration; and at least one potent solvent in a sufficient amount to substantially solubilize the at least one active agent in the composition. The term “substantially soluble” means that at least 95% of the active agent has been solubilized, i.e., 5% or less of the active agent is present in a solid state. In one or more embodiments, the concentration of the at least one potent solvent is more than about 40% of the at least one solvent of the composition; or even more than about 60%.

Non-limiting examples of pairs of active agent and potent solvent include: Betamethasone valerate: Practically insoluble in mineral oil (<0.01%); soluble more than 1% in glycofurol; Hydrocortisone butyrate: Practically insoluble in mineral oil (<0.01%); soluble more than 1% in glycofurol; Metronidazole: Practically insoluble in mineral oil (<0.01%); soluble more than 1% in dimethyl isosrbide; Ketoconazole: Practically insoluble in mineral oil (<0.01%); soluble more than 1% in glycofurol, propylene glycol and dimethyl isosrbide; Mupirocin: Practically insoluble in mineral oil (<0.01%); soluble more than 1% in glycofurol, hexylene glycol, dimethyl isosorbide, propylene glycol and polyethylene glycol 400 (PEG 400); Meloxicam, a nonsteroidal anti-inflammatory agent: Practically insoluble in mineral oil (<0.001%); soluble in propylene glycol: 0.3 mg/mL; and in PEG 400: 3.7 mg/mL; and Progesterone: Practically insoluble in mineral oil (<0.001%); soluble in PEG 400: 15.3 mg/mL.

A non-limiting exemplary list of solvents that can be considered as potent solvents includes polyethylene glycol, propylene glycol, hexylene glycol, butaneediols and isomers thereof, glycerol, benzyl alcohol, DMSO, ethyl oleate, ethyl caprylate, diisopropyl adipate, dimethylacetamide, N-methylpyrrolidone, N-hydroxyethylpyrrolidone, polyvinylpyrrolidone, isosorbide derivatives, such as dimethyl isosorbide, glycofurol and ethoxydiglycol (transcutol) and laurocapram.

The use of a potent solvent in a foam composition provides an improved method of delivering poorly soluble therapeutic agents to a target area. It is known that low drug solubility results in poor bioavailability, leading to decreased effectiveness of treatment. Foam compositions, for which the solvent includes a potent solvent, increase the levels of the active agent in solution and thus, provide high delivery and improved therapy.

Potent solvents, as defined herein, are usually liquid. Formulations comprising potent solvents and active agents are generally disadvantageous as therapeutics, since their usage involves unwanted dripping and inconvenient method of application; resulting in inadequate dosing. Surprisingly, the foams, which are drip-free, provide a superior vehicle for such active agents, enabling convenient usage and accurate effective dosing.

In one or more embodiments, the foamable pharmaceutical composition may additionally include a mixture of two or more of the solvents selected from the group of hydrophobic solvents, silicone oils, emollients, polar solvents and potent solvents in an appropriate proportion as would be appreciated to a person skilled in the art.

In one or more embodiments, the PPG alkyl ether may act as a potent solvent

### Additional Components

In an embodiment, a composition includes one or more additional components. Such additional components include but are not limited to anti-perspirants, anti-static agents, buffering agents, bulking agents, chelating agents, cleansers, colorants, conditioners, deodorants, diluents, dyes, emollients, fragrances, hair conditioners, humectants, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers and vitamins. As is known to one skilled in the art, in some instances a specific additional component may have more than one activity, function or effect.

### Propellants

Suitable propellants include volatile hydrocarbons such as butane, propane, isobutane and fluorocarbon gases, or mixtures thereof.

In an embodiment the propellant is AP 70 which is a mixture of propane, isobutene and butane.

The propellant makes up about 5-25 wt % of the foamable composition. In some circumstances the propellant is present in the composition at a concentration of up to 35%. In some embodiments, the propellant is about 3% to about 25% by weight of the composition. The propellants are used to generate and administer the foamable composition as a foam. The total composition including propellant, foamable carrier or vehicle, and optional ingredients is referred to as the foamable composition. The percentage of propellant in the foamable composition is calculated relative to the total amount of foamable carrier or vehicle, as well as optional ingredients. Accordingly the relative amount of propellant to all of the other components of the foamable composition (e.g., glycerol ether, stabilizer, water, etc.) is alternatively expressed as a weight ratio of propellant to vehicle/carrier, where the ratio of propellant to vehicle/carrier ranges from about 5:100 to about 25:100. In some embodiments, the ratio of propellant to vehicle/carrier is about 3:100 to about 25:100. In some embodiments, the ratio of propellant to vehicle/carrier is not more than about 3:100 to about 15:100. In some embodiments, the ratio of propellant to vehicle/carrier is about 5:100 to about 15:100. In some embodiments, the ratio of propellant to vehicle/carrier is not more than about 35:100. For example propellant calculations, see the Examples.

Alcohol and organic solvents render foams inflammable. It has been surprisingly discovered that fluorohydrocarbon propellants, other than chloro-fluoro carbons (CMCs), which are non-ozone-depleting propellants, are particularly useful in the production of a non-flammable foamable composition. A test according to European Standard prEN 14851, titled “Aerosol containers—Aerosol foam flammability test” revealed that compositions containing an organic carrier that contains a hydrophobic organic carrier and/or a polar solvent, which are detected as inflammable when a hydrocarbon propellant is used, become non-flammable, while the propellant is an HFC propellant.

Such propellants include, but are not limited to, hydrofluorocarbon (HFC) propellants, which contain no chlorine atoms, and as such, fall completely outside concerns about stratospheric ozone destruction by chlorofluorocarbons or other chlorinated hydrocarbons. Exemplary non-flammable propellants according to this aspect include propellants made by DuPont under the registered trademark Dymel, such as 1,1,1,2 tetrafluorethane (Dymel 134), and 1,1,1,2,3,3,3 heptafluoropropane (Dymel 227). HFCs possess Ozone Depletion Potential of 0.00 and thus, they are allowed for use as propellant in aerosol products.

Notably, the stability of foamable emulsions including HFC as the propellant can be improved in comparison with the same composition made with a hydrocarbon propellant.

In one or more embodiments the foamable compositions comprise a combination of a HFC and a hydrocarbon propellant such as n-butane or mixtures of hydrocarbon propellants such as propane, isobutane and butane.

### Microemulsions and Nanoemulsions

In an embodiment the glycerol ether composition is an emulsion. In a particular embodiment the composition is a microemulsion or a nanoemulsion, which are monophasic, transparent (or slightly translucent) dispersions of oil and water capable of maintaining stability for substantial periods of time. They and a method of manufacture are more particularly described in U.S. application Ser. No. 11/389,742, filed Mar. 27, 2006, published on Oct. 19, 2006 as US2006/0233721, entitled “FOAM CONTAINING UNIQUE OIL GLOBULES”; and co-pending and commonly owned application U.S. application Ser. No. 11/975,621, filed on Oct. 19, 2007, published as US2008/0138296 on Jun. 12, 2008, and entitled “FOAM PREPARED FROM NANOEMULSIONS AND USES,” which are incorporated herein by way of reference in their entirety. As will be appreciated by one of skill in the art the methodology may be adapted according to the type of carrier composition. Microemulsion or nanoemulsion ether formulations may improve the delivery and penetration of an active agent in the skin. In general terms the composition is homogenized to obtain a fine emulsion, which is further finely dispersed using high pressure homogenizer (Model M-110Y Microfluidezer® processor, Microfluidics Corp, USA) at 1000-1500 bars pressure, e.g. 2 to 8 cycles to achieve the appropriate size.

### Composition and Foam Physical Characteristics and Advantages

A pharmaceutical or cosmetic composition manufactured using the foamable carrier is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed. It can in an embodiment also be simply applied to a specific area without any mechanical application to deposit non-volatile constituents.

The foamable composition forms fine bubble structures that spread easily on the treated area and absorb quickly.

The composition should also be free flowing, to allow it to flow through the aperture of the container, e.g., an aerosol container, and create an acceptable foam.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

Another property of the foam is specific gravity, as measured upon release from the aerosol can. Typically, foams have specific gravity of less than 0.12 g/mL; or less than 0.10 g/mL; or less than 0.08 g/mL, depending on their composition and on the propellant concentration.

### Pharmaceutical Composition

The foamable carrier is an ideal vehicle for active pharmaceutical ingredients and active cosmetic ingredients. In this context, active pharmaceutical ingredients and active cosmetic ingredients are collectively termed “active agent” or “active agents.” The active agents can be used in the formulation as a suspended solid or in solution, alone or in combination with other active agents.

In one or more embodiments the active ingredient is an anti-cancer or an anti-lesion or an anti-leishmaniosis agent. In one or more embodiments reactive ingredient is a phosphocholine or an alkyl phosphoric acid such as hexadecylphosphocholine.

In one or more embodiments the active ingredient is an immune response modifier or antiviral agent selected from the group consisting of imiquimod, resiquimod, and gardiquimod.

Imiquimod (4-Amino-1-isobutyl-1H-imidazo[4,5-c]quinoline) an imidazoquinoline amine, is an immune response modifier used topically in the treatment of external genital and perianal warts, superficial basal cell carcinomas, and actinic keratoses. It is applied as a 5% cream, with the frequency and period varying depending on the disease. Imiquimod is under investigation for the treatment of Bowen's disease and of other squamous cell carcinomas. Adverse effects after topical application of imiquimod include local skin erosion, erythema, excoriation, flaking, and oedema. There have been reports of localised hypopigmentation and hyperpigmentation. Skin reactions away from the site of application have been reported. Imiquimod is used topically for the treatment of external genital and perianal exophytic warts (condylomata acuminata) caused by human papillomavirus (HPV). Potentially it may have application for treating body cavity warts, urethral, intravaginal, cervical, rectal, intra-anal, or oral HPV warts. Imiquimod has been used topically for the treatment of molluscum contagiosum and for the treatment of actinic keratoses. It may also have potential for the topical treatment of verruca vulgaris (common warts). Alternative regimens include intralesional interferon alfa and laser surgery. There is some evidence that warts located on moist surfaces and/or in intertriginous areas appear to respond better to topical treatments. On this basis in one or more embodiments, the imiquimod formulation further comprises a moisturiser and/or a humectant.

Other members of the imiquimod family are resiquimod (used against herpes simplex virus infections, including genital herpes and is a potential treatment of various other diseases, including other viral infections and eczema and as a vaccine adjuvant) and gardiquimod (a potential antiviral).

In one or more embodiments, imiquimod is the active ingredient. It can be used in the formulation as a suspended solid or in solution, alone or in combination with other active agents. As is known to one skilled in the art, in some instances a specific active agent may have more than one activity, function or effect.

In one or more embodiments the active agent is an antineoplastic agent. Non-limiting examples include nitrogen mustard analogues, alkyl sulfonates, ethylene imines, nitrosoureas, epoxides, other, alkylating agents, folic acid analogues, purine analogues, pyrimidine analogues, vinca alkaloids and analogues, podophyllotoxin derivative, colchicine derivatives, taxanes, other plant alkaloids and natural products, actinomycines, anthracyclines and related substances, other cytotoxic antibiotics, platinum compounds, methylhydrazines, monoclonal antibodies, agents used in photodynamic therapy, amsacrine, asparaginase, altretamine, hydroxycarbamide, lonidamine, pentostatin, miltefosine, masoprocol, estramustine, tretinoin, mitoguazone, topotecan, tiazofurine, irinotecan, alitretinoin, mitotane, pegaspargase, bexarotene, arsenic trioxide, imatinib, denileukin diftitox, gefitinib, bortezomib, and celecoxib.

In one or more embodiments the active agent is chemically similar to hexadecylphosphocholine. Non-limiting examples include n-octylphosphorylcholine, n-octadecylphosphocholine, n-decylphosphorylcholine, n-tetradecylphosphorylcholine, choline, hydroxide, 3-(dodecyloxy)propyl hydrogen phosphate, inner salt, erucylphosphocholine, compound with m 18498-26-5, oleyloxyethylphosphorylcholine, 2-tetradecyloctadecano(1)phosphocholine, 2-n-propyl-platelet activating factor, 3,5-dioxa-8-thia-4-phosphatetracosan-1-aminium, 4-hydroxy-n,n,n-trimethyl-, inner salt, 4-oxide.

Suitable active agents for use in the vehicles and formulations described herein alone or in conjunction with a phosphocholine or an alkyl phosphoric acid such as hexadecylphosphocholine include, but are not limited to, active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, dicarboxylic acids, disinfectants, fungicides, hair growth regulators, hormones, hydroxy acids, interferons, immunosuppressants, immunoregulating agents, insecticides, insect repellents, keratolytic agents, lactams, metals, metal oxides, mitocides, neuropeptides, steroids, non-steroidal anti-inflammatory agents, oxidizing agents, pediculicides, photodynamic therapy agents, retinoids, sanatives, scabicides, self tanning agents, skin whitening agents, vasoconstrictors, vasodilators, vitamins, vitamin D derivatives, wound healing agents and wart removers as well as agents having activity against superficial basal cell carcinomas, actinic keratoses, Bowen's disease and/or other squamous cell carcinomas and molluscum contagiosum. As is known to one skilled in the art, in some instances a specific active agent may have more than one activity, function or effect.

In one or more embodiments, the formulation includes a steroidal anti-inflammatory agent. Exemplary steroidal anti-inflammatory agents include, but are not limited to, corticosteroids such as hydrocortisone, hydroxyltriamcinolone, alpha-methyl dexamethasone, dexamethasone-phosphate, beclomethsone dipropionate, clobetasol valemate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortmate, mepreddisone, paramethasone, prednisolone, prednisone, beclomethasone dipropionate, triamcinolone, and mixtures thereof.

In one embodiment, the formulation includes an immunomodulator. Immunomodulators are chemically or biologically-derived agents that modify the immune response or the functioning of the immune system (as by the stimulation of antibody formation or the inhibition of white blood cell activity). Immunomodulators include, among other options, cyclic peptides, such as cyclosporine, tacrolimus, tresperimus, pimecrolimus, sirolimus (rapamycin), verolimus, laflunimus, laquinimod and imiquimod. Such compounds, delivered in the foam, are especially advantageous in skin disorders such as psoriasis, eczema and atopic dermatitis, where the large skin areas are to be treated.

In an embodiment, the active agent is selected from at least one of imiquimod, an interferon, an immodulator, podophyllin (anti-mitotic), podofilox, 5-fluorouracil (5-FU), and trichloroacetic acid (TCA).

In an embodiment, the active agent is selected from at least one of Fluorouracil, afovirsen, inosine pranobex, podophyllum, trichloroacetic acid, thiotep, diclofenac, 5-aminolevulinic acid and derivatives, and tretinoin.

In one or more embodiments hexadecylphosphocholine is used in combination with meglumine antimoniate (for cutaneous leishmaniasis)

In one or more embodiments imiquimod is used in combination with meglumine antimoniate (for cutaneous leishmaniasis); cryotherapy [liquid nitrogen](for plantal and periungual warts/actinic keratoses); acyclovir (genital hsv-2 infection); 5-aminolevulinic acid (for genital bowenoid papulosis); fluorouracil (anal and perianal squamous cell carcinoma; salicylic acid (anal and genital warts); a COX inhibitor sulindac (for squamous cell carcinoma).

In one or more embodiments the formulation is resistant to oxidation of the active agent. Resistance may be due to one or more features. It may be inherently resistant. Resistance may be conferred by one or more agents such as antioxidants, flavanoids, pH stabilizers and adjusters, modulating agents, the absence of oxygen and/or presence of propellant in the canisters and/or during manufacture.

Suitable buffering agents include but are not limited to acetic acid, adipic acid, calcium hydroxide, citric acid, glycine, hydrochloric acid, lactic acid, magnesium aluminometasilicates, phosphoric acid, sodium carbonate, sodium citrate, sodium hydroxide, sorbic acid, succinic acid, tartaric acid, and derivatives, salts and mixtures thereof. A preferred buffer is citric acid and sodium hydroxide.

In one or more embodiments the active agent comprises a flavonoids (or bioflavonoids) are a large group of polyphenolic antioxidant compounds, which often occur as glycosides and are ubiquitously present in foods of plant origin. Some flavonoids (e.g. quercetin, rutin) are available as dietary supplements. Flavonoids can be further subdivided into:
flavonols (e.g. kaempferol, quercetin and myricetin)
flavones (e.g. apigenin and luteolin)
flavonones (e.g. hesperetin, naringenin, eriodictyol)
flavan-3-ols (e.g. (+)-catechin, (+)-gallocatechin, (−)-epicatechin, (−)-epigallocatechin)
anthocyanins (e.g. cyanidin, delphinidin, malvidin, pelargonidin, peonidin, petunidin)
proanthocyanidins.

### Fields of Applications

The foamable carrier is suitable for treating any inflicted surface. In one or more embodiments, foamable carrier is suitable for administration to the skin, a body surface, a body cavity or mucosal surface, e.g., the cavity and/or the mucosa of the, ear, vagina or rectum (severally and interchangeably termed herein “target site”).

In one embodiment, the disorder is a dermatological disorder, which can be treated by an active agent or can prevent or ameliorate the disorder. In another embodiment the disorder is a mucosal disorder.

In another embodiment, the disorder is a dermatological disorder that benefits from the use of phosphocholine or alkyl phosphoric acid such as hexadecylphosphocholine alone or in conjunction with another active agent. The ether may be of benefit by improving the solubility of the active agent or increasing the penetration of the active agent. The ether may also provide a synergistic therapeutic effect in combination with the active agent.

By selecting a suitable active agent, or a combination of two or more active agents, the foamable composition is useful in treating an animal or a human patient having any one of a variety of dermatological disorders, including dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, eethyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, granuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo.

Likewise, the foamable composition is suitable for treating a disorder of a body cavity or mucosal surface, e.g., the mucosa of the nose, mouth, eye, ear, respiratory system, vagina or rectum. Non-limiting examples of such conditions include chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranuloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

In an embodiment, the disorder is a dermatological disorder, which can be treated, ameliorated or prevented by an ether.

In an embodiment, the disorder is a dermatological disorder, which can be treated, ameliorated or prevented by at least one of imiquimod, an interferon, an immodulator, podophyllin (anti-mitotic), podofilox, 5-fluorouracil (5-FU), and trichloroacetic acid (TCA).

In an embodiment, the disorder is a dermatological disorder, which can be treated by a topical steroid.

In an embodiment, the disorder is a dermatological disorder, which can be treated by a coal tar alone or in combination with another active agent such as a steroid.

In an embodiment, the disorder is a dermatological disorder, which can be treated by an immunomodulator.

In an embodiment, the disorder is a dermatological disorder, which can be treated by an anti-infective agent, such as an antibacterial agent, and antibiotic, an antifungal agent and an antiviral agent.

In an embodiment, the disorder is a dermatological disorder, which is common in children. Foam is advantageous in the topical treatment of children, who are sensitive to treatment with a cream or ointment.

In an embodiment, the disorder is atopic dermatitis and the active agent is a steroid.

In an embodiment, the disorder is psoriasis and the active agent is a steroid, optionally further including a dicarboxylic acid (DCA) or DCA ester to stabilize or solubilize the topical steroid.

In an embodiment, the disorder is selected from psoriasis and atopic dermatitis and the active agent comprises a steroid and an additional non-steroidal active agent, such as a vitamin D derivative, optionally further including a DCA or DCA ester to stabilize or solubilize the topical steroid and/or non-steroidal active agent.

In an embodiment, the disorder is selected from psoriasis and atopic dermatitis and the active agent comprises an immunomodulator, optionally further including a DCA or DCA ester to stabilize or solubilize the immunomodulator.

In an embodiment, the composition is useful for the treatment of an infection. In one or more embodiments, the composition is suitable for the treatment of an infection, selected from the group of a bacterial infection, a fungal infection, a yeast infection, a viral infection and a parasitic infection.

In an embodiment, the composition is useful for the treatment of wound, ulcer and burn.

The composition is also suitable for administering a hormone to the skin or to a mucosal membrane or to a body cavity, in order to deliver the hormone into the tissue of the target organ, in any disorder that responds to treatment with a hormone.

Other foamable compositions are described in: U.S. Publication No. 05-0232869, published on Oct. 20, 2005, entitled NONSTEROIDAL IMMUNOMODULATING KIT AND COMPOSITION AND USES THEREOF; U.S. Publication No. 05-0205086, published on Sep. 22, 2005, entitled RETINOID IMMUNOMODULATING KIT AND COMPOSITION AND USES THEREOF; U.S. Publication No. 06-0018937, published on Jan. 26, 2006, entitled STEROID KIT AND FOAMABLE COMPOSITION AND USES THEREOF; U.S. Publication No. 05-0271596, published on Dec. 8, 2005, entitled VASOACTIVE KIT AND COMPOSITION AND USES THEREOF; U.S. Publication No. 06-0269485, published on Nov. 30, 2006, entitled ANTIBIOTIC KIT AND COMPOSITION AND USES THEREOF; U.S. Publication No. 07-0020304, published on Jan. 25, 2007, entitled NON-FLAMMABLE INSECTICIDE COMPOSITION AND USES THEREOF; U.S. Publication No. 06-0193789, published on Aug. 31, 2006, entitled FILM FORMING FOAMABLE COMPOSITION; U.S. Publication No. 2007-0292355 published on Dec. 20, 2007 and entitled ANTI-INFECTION AUGMENTATION OF FOAMABLE COMPOSITIONS AND KIT AND USES THEREOF; U.S. Publication No. 2008-0069779 and entitled DICARBOXYLIC ACID FOAMABLE VEHICLE AND PHARMACEUTICAL COMPOSITIONS THEREOF; U.S. Publication 20080206159, published on Aug. 28, 2008 and entitled COMPOSITIONS WITH MODULATING AGENTS; U.S. patent application Ser. No. 11/767,442, filed on Jun. 22, 2007, entitled FOAMABLE COMPOSITIONS AND KITS COMPRISING ONE OR MORE OF A CHANNEL AGENT, A CHOLINERGIC AGENT, A NITRIC OXIDE DONOR, AND RELATED AGENTS AND THEIR USES; U.S. Publication 2008-0069779, published on Mar. 20, 2008 and entitled FOAMABLE VEHICLE AND VITAMIN AND FLAVONOID PHARMACEUTICAL COMPOSITIONS THEREOF, all of which are incorporated herein by reference in their entirety. More particularly any of the active ingredients; the solvents; the surfactants; foam adjuvants; penetration enhancers; humectants; moisturizers; and other excipients as well as the propellants listed therein can be applied herein and are incorporated by reference.

The following examples further exemplify the foamable pharmaceutical carriers, pharmaceutical compositions thereof, methods for preparing the same, and therapeutic uses of the compositions described herein. The examples are for the purposes of illustration only and are not intended to be limiting. Many variations may be carried out by one of ordinary skill in the art and are contemplated within the full scope.

### Methodology

A general procedure for preparing foamable compositions is set out in WO 2004/037225, which is incorporated herein by reference in its entirety.

### Ether Phase

In a closed environment add liquid ethers to the water phase and mix. The water phase may be water alone or may contain a buffer system and/or modulating agent.

### Stabilizing Phase

1) Water and polymer if any are placed in container at room temperature and pressure and mixed using a high shear force homogenizer. After about 10 minutes, surfactant is added. Mixing is continued for about an additional 5 minutes. If required the ingredients can be heated to 60-75 degrees C. to complete dissolution. Cool to room temperature while mixing.

### Single Phase Foam

Stock ether solution is then added to the stabilizing water phase preferably in an aluminum canister in a closed environment and is crimped immediately with a valve to seal the composition to avoid evaporation. After shaking for 2 minutes, propellant is added through the valve and canister is subjected to shaking.

### Emulsion Foam

A. Prepare Emulsion Phase.
1. Mix oily phase ingredients and heat to 75° C. to melt all ingredients and obtain a homogeneous mixture.
2. Mix polymers in water with heating or cooling as appropriate for specific polymer. While the polymers may be added instead into the oily phase it was found to be advantageous to prepare them in the water phase.
3. Add all other water soluble ingredients to water-polymer solution and heat to 75° C.
4. Slowly add the internal phase to the external phase at 75° C. under vigorous mixing and homogenize to obtain a fine emulsion. Alternatively the external phase is added slowly to the internal phase.
5. Cool to below 40° C. and add sensitive ingredients with mild mixing.
6. Cool to room temperature.

B. Add Ether Phase
7. Stock ether solution is then added to the emulsion phase preferably in an aluminum canister and is crimped immediately with a valve to seal the composition to avoid evaporation. After shaking for 2 minutes, propellant is added through the valve and the canister is subjected to shaking.

### Substantially Waterless Foam

A. Prepare Substantially Waterless Phase
1. Dissolve the polymers in the main solvent with heating or cooling as appropriate for specific polymer. Add all the other ingredients and heat to 75° C. to melt and dissolve the various ingredients.
2. Cool to below 40° C. and add sensitive ingredients with mild mixing.
3. Cool to room temperature.

B. Add Ether Phase
4. Stock ether solution is then added to the substantially waterless phase preferably in an aluminum canister and is crimped immediately with a valve to seal the composition to avoid evaporation. After shaking for 2 minutes, propellant is added through the valve and the canister is subjected to shaking.

Canisters Filling and Crimping

Each aerosol canister is filled with PFF and crimped with valve using vacuum crimping machine.

Pressurizing

Propellant Filling

Pressurizing is carried out using a hydrocarbon gas or gas mixture

Canisters are filled and then warmed for 30 sec in a warm bath at 50° C. and well shaken immediately thereafter.

Closure Integrity Test.

Each pressurized canister is subjected to bubble and crimping integrity testing by immersing the canister in a 60° C. water bath for 2 minutes. Canisters are observed for leakage as determined by the generation of bubbles. Canisters releasing bubbles are rejected.

### Stock Compositions

Non-limiting examples of how stock solutions are made up with and without API. Other stock solutions may be made using the same methodology by simply varying adding or omitting ingredients as would be appreciated by one of ordinary skill in the art.

### EXAMPLES

The foamable composition is described with reference to the following examples. This foamable composition is not limited to these examples and experiments. Many variations will suggest themselves and are within the full intended scope of the appended claims.

### Section A—Glycerol Ether Formulations

### A1—Example 1

### Compositions Containing ˜61% Glycerol Ethers and an Active Pharmaceutical Ingredient

The following foamable compositions were prepared and the quality of the resultant foam was ascertained.

### a) Formulations

| Lot: | A1 | A2 |
|---|---|---|
| Ingredient | W/W % | W/W % |
| Hydroxypropylcellulose EF | 0.50 | — |
| Polysorbate 80 | 2.00 | — |
| Stock Ether Solution | 97.50 | 100 |
| Total | 100.00 | 100.00 |
| Propellant (AP-70) | 10.00 | 10.00 |

### b) Stock Ether Solution

Ingredient

| Stock Ether Solution | W/W % |
|---|---|
| Hexadecylphosphocholine | 6.00 |
| Glycerol propyl ether | 31.30 |
| Glycerol hexyl ether | 15.70 |
| Glycerol nonyl ether | 15.70 |
| Water, purified, buffered | 31.30 |
| Total | 100 |

### c) Results

| Results | Lot A1 | Lot A2 |
|---|---|---|
| Foam quality | G-FG | F |
| Color | white | white |
| Odor | very faint | non |
| Shakability | moderate | moderate |
| Noise | Yes | Yes |
| Controlled dispersing | Yes | Yes |
| pH with water 1:5 | 5.83 | N/R |
| Microscopic observation | no crystals | no crystals |

G-FG = Good-Fairly Good

These data demonstrate that compositions containing 61%, glycerol ethers to provide a unique foam delivery system, which is (1) substantially stable and (2) breakable to allow ease of application; and provides (3) solubilizing capacity; and (4) potentially enhanced skin delivery of an active agent (5) enabling both shakability and flowability of the wax formulation and potentially enhanced skin penetration. Although glycerol ethers are amphiphilic solvents which can have some surfactant-like properties (e.g., reduction of interfacial tensions) they do not generate foams of quality alone even at very high concentrations.

Additional agents are required to form good quality foam. While a foam of quality was achieved by the addition of a liquid non-ionic surfactant in combination with a cellulose polymer it is anticipated that increasing the polymer and/or surfactant can further improve the foam quality. However, the behavior of glycerol ethers is non-obvious when combined with water and propellant and ultimately expelled from a canister. Thus discovering an effective balance between the amount of volatile ethers and the amount of stabilizers and other ingredients to achieve a breakable and thermolabile foam of quality is non-obvious.

The compositions contain significant levels of water, which can contribute to a good skin feeling effect even at high ether concentrations.

These compositions can be used as a vehicle for pharmaceutical and cosmetic agents especially those soluble in ethers or water.

The compositions may be buffered with a suitable buffer. In the above example the buffer is part of the aqueous phase and is a combination of citric acid (˜0.48% of 100%) and sodium hydroxide (˜0.23% of 100%). A mildly acidic pH within the range of that of skin is preferred.

In order to create a foamable composition, the composition is filled into an aerosol canister and pressurized using a liquefied or gas propellant added at a concentration of about 3% to about 25%.

### A2—Example 2

### Composition Containing ˜29% Glycerol Ethers

The following foamable pharmaceutical compositions were prepared and the quality of the resultant foam was ascertained.

### a) Formulation

| Ingredient | A3 (w/w %) |
|---|---|
| Water | 50.00 |
| Avicel 581RC | 2.00 |
| Polysorbate 80 | 2.00 |
| Stock Ether Solution | 46.00 |
| Total | 100.00 |
| Propellant (AP-70) | 8.00 |

### b) Stock Ether Solution

Ingredient

| Stock Ether Solution | W/W % |
|---|---|
| Hexadecylphosphocholine | 6.00 |
| Glycerol propyl ether | 31.30 |
| Glycerol hexyl ether | 15.70 |
| Glycerol nonyl ether | 15.70 |
| Water, purified, buffered | 31.30 |
| Total | 100 |

### c) Preparation:

Water and avicel were placed in aluminum canister at room temperature and pressure and mixed using a high shear force homogenizer. After about 10 minutes, polysorbate 80 was added. Mixing then continued for about an additional 5 minutes. Stock ether solution was then added to the stabilizing water phase and crimped immediately with a valve to seal the composition to avoid evaporation. After shaking for 2 minutes, propellant was added through the valve and the canister was subjected to shaking.

### d) Results

| Results | A3 |
|---|---|
| Foam Quality | Good to Excellent |
| Color | White |
| Odor | No |
| Density | 0.040 |
| Collapse Time | 60/P |
| Bubble Size (μm) | 173 |
| Bubble Size (Above 500 μm) | 0.0 |
| Microscope | single phase |

These compositions contain about 29% of a mixture of high, medium and low glyceryl ethers and about 64% water a unique foam delivery system, which is (1) substantially stable and (2) breakable thermolabile to allow ease of application; and provides (3) solubilizing capacity; and (4) potentially enhanced skin delivery of an active agent (5) enabling both shakability and flowability of the ether formulation and potentially enhanced skin penetration.

The behavior of glycerol ethers is non-obvious when combined with water and propellant and ultimately released from a canister. Thus discovering an effective balance between the amount of volatile ethers and the amount of stabilizers and other ingredients to achieve a breakable and thermolabile foam of quality is non-obvious. Likewise selecting ingredients so that there is a single phase without separation is non-obvious.

The compositions contain about 64% water. Therefore, they can provide a mild skin feeling effect.

The formulations may be buffered with a suitable buffer. In the above example the buffer is part of the aqueous phase and is a combination of citric acid (˜0.48% of 100%) and sodium hydroxide (˜0.23% of 100%). A mildly acidic pH within the range of that of skin is preferred.

The composition is a single phase.

The compositions can be used for topical therapy of a skin disorder treatable by hexadecylphosphocholine, and similar agents which can include treating, containing, ameliorating or preventing topical and skin lesions, cancer and skin cancer, dermatitis and leishmaniosis.

These compositions can be used as a vehicle for other pharmaceutical and cosmetic agents especially those soluble in volatile ethers or water. These compositions are thermolabile. Although they produce a foam which initially appears to be breakable, as the formulation warms up it evaporates rather than collapses. Non-volatile ethers may be added to alter the thermolability, the rate of evaporation, and skin feeling.

### A3-A8—Example 3

### Compositions Containing ˜60% to ˜63% Glycerol Ethers, with Poloxamer or Cellulose or Both and an Active Pharmaceutical Ingredient

### a) Stock Ether Solution

Ingredient

| Stock Ether Solution | W/W % |
|---|---|
| Hexadecylphosphocholine | 6.00 |
| Glycerol propyl ether | 31.30 |
| Glycerol hexyl ether | 15.70 |
| Glycerol nonyl ether | 15.70 |
| Water, purified, buffered | 31.30 |
| Total | 100 |

The formulations may be buffered with a suitable buffer. In the above example the buffer is part of the aqueous phase and is a combination of citric acid (˜0.48% of 100%) and sodium hydroxide (˜0.23% of 100%). A mildly acidic pH within the range of that of skin is preferred.

In order to create a foamable composition, the composition is filled into an aerosol canister and pressurized using a liquefied or gas propellant can be added at a concentration of about 3% to about 25%.

b) Formulations with Poloxamer

| | A3 | A4 |
|---|---|---|
| | W/W % | W/W % |
| Ingredient | | |
| Poloxamer 407 | 5.00 | 5.00 |
| Stock ether solution | 95.00 | — |
| Water, purified | — | 95.00 |
| Total | 100.00 | 100.00 |
| Propellant (AP-70) | 10.00 | 10.00 |
| Results | | |
| Foam quality | Fair | Fair |
| Color | white | white |
| Odor | no | no |
| Shakability | moderate | moderate |

Procedure: Combine ingredients together and place in refrigerator at 10° C. until Poloxamer is dissolved. Place the PFF into a canister and crimp with valve. Fill with the propellant.

Although glycerol ethers are amphiphilic solvents which can have some surfactant-like properties (e.g., reduction of interfacial tensions) they alone do not generate foams of quality even at very high concentrations.

Additional stabilizing agents are required to form good quality foam. While a foam of quality can be achieved by the addition of a non-ionic surfactant, the behavior of glycerol ethers is non-obvious when combined with water and propellant. A polymeric surfactant, poloxamer 407 alone, even at 5% concentration does not generate a foam of any quality. Thus discovering an effective balance between the amount of volatile ethers and the amount of stabilizers and other ingredients to achieve a breakable and thermolabile foam of quality is non-obvious.

c) Formulations with Hydroxyethylcellulose (HEC) as Polymer

| | A5 | A6 |
|---|---|---|
| | W/W % | W/W % |
| Ingredient | | |
| Hydroxyethylcellulose (HEC) | 1.00 | 1.00 |
| Stock ether solution | 99.00 | — |
| Water, purified | — | 99.00 |
| Total | 100.00 | 100.00 |
| Propellant (AP-70) | 10.00 | 10.00 |
| Results | | |
| Foam quality | Fair | Fair/bubble gel |
| Color | white | white |
| Odor | no | no |
| Shakability | moderate | moderate |

Procedure: Combine ingredients. Dissolve HEC using Rotor-Stator homogenizer. Place the PFF into a canister and crimp with valve. Fill with the propellant.

The behavior of glycerol ethers is non obvious when combined with water and propellant and a cellulose polymer. Hydroxyethylcellulose (HEC) alone, at 1% concentration does not generate a foam of any quality. Thus discovering an effective balance between the amount of volatile ethers and the amount of stabilizers and other ingredients to achieve a breakable and thermolabile foam of quality is non obvious.

d) Formulations with Hydroxyethylcellulose (HEC) as Polymer and Poloxamer as a Surfactant Polymer

| | A7 | A8 |
|---|---|---|
| | W/W % | W/W % |
| Ingredient | | |
| Hydroxyethylcellulose (HEC) | 1.00 | 1.00 |
| Poloxamer 407 | 5.00 | 5.00 |
| Stock ether solution | 94.00 | — |
| Water | — | 94.00 |
| Total | 100.00 | 100.00 |
| Propellant (AP-70) | 10.00 | 10.00 |
| Results | | |
| Foam quality | Fair | Excellent |
| Breakable | Quick break | Stable breakable |
| | collapses rapidly | |
| Color | white | white |
| Odor | no | no |
| Shakability | moderate | moderate |

Procedure: Combine ingredients together and place in refrigerator at temp 10° C. until Poloxamer is dissolved. Add and dissolve H EC using Rotor-Stator homogenizer. Place the PFF into a canister and crimp with valve. Fill with the propellant.

The behavior of glycerol ethers is non obvious when combined with water and propellant, polymeric surfactant 5% and a cellulose polymer. Hydroxyethylcellulose (HEC) alone at 1% concentration does not generate a foam of any quality. Poloxamer alone at 5% concentration does not generate a foam of any quality. Hydroxyethylcellulose (HEC) at 1% concentration plus poloxamer at 5% concentration, however does generate excellent quality foam that is stable but breakable on shear force. Nevertheless, when this successful cellulose and poloxamer combination is incorporated with glycerol ethers this composition surprisingly does not generate a foam of any quality. Thus discovering an effective balance between the amount of volatile ethers and the amount of stabilizers and other ingredients to achieve a breakable and thermolabile foam of quality as seen in Examples 1 and 2 is non-obvious.

### Section B—Prophetic Carrier Formulations

### B1 Example 4

### Prophetic Vehicle Compositions Containing Glycerol Propyl Ether; Glycerol Hexyl Ether; Glycerol Nonyl Ether

The following foamable compositions can be prepared as detailed in example 2.

### a) Stock

| Ingredients for Stock | B1 | B2 | B3 | B4 | B5 | B6 |
|---|---|---|---|---|---|---|
| Ether Solution | (w/w %) | (w/w %) | (w/w %) | (w/w %) | (w/w %) | (w/w %) |
| Glycerol propyl ether | 32.00 | 63.4 | 50.00 | 32.00 | 32.00 | 32.00 |
| Glycerol hexyl ether | 15.70 | | | | 31.40 | |
| Glycerol nonyl ether | 15.70 | | | | | 31.40 |
| Water, purified, buffered | 36.60 | 36.60 | 50.00 | 68.00 | 36.60 | 36.60 |
| Total | 100 | 100 | 100 | 100 | 100 | 100 |

### b) ˜29% to ˜15% Formulations

| Ingredient | W/W % |
|---|---|
| Water | 50.00 |
| Avicel 581RC | 2.00 |
| Polysorbate 80 | 2.00 |
| Stock Ether Solution | 46.00 |
| Total | 100.00 |
| Propellant (AP-70) | 8.00 |

### c) ˜61% to ˜31% Formulations

| Ingredient | W/W % |
|---|---|
| Hydroxypropylcellulose EF | 1.00 |
| Polysorbate 80 | 2.00 |
| stock ether solution | 97.00 |
| total | 100.00 |
| propellant (AP-70) | 8.00 |

The compositions may be buffered with a suitable buffer. In the above example the buffer is part of the aqueous phase and is a combination of citric acid (˜0.48% of 100%) and sodium hydroxide (˜0.23% of 100%). A mildly acidic pH within the range of that of skin is preferred.

### B2 Example 5

### Prophetic Vehicle Compositions Containing One or More of Glycerol Propyl Ether; Glycerol Hexyl Ether; Glycerol Nonyl Ether with Foam Adjuvants

The following foamable compositions can be prepared as detailed in example 2.

### a) Stock

| Ingredients for Stock | B7 | B8 | B9 | B10 | B11 | B12 |
|---|---|---|---|---|---|---|
| Ether Solution | (w/w %) | (w/w %) | (w/w %) | (w/w %) | (w/w %) | (w/w %) |
| Glycerol propyl ether | 32.00 | 63.4 | 50.00 | 32.00 | 32.00 | 32.00 |
| Glycerol hexyl ether | 15.70 | | | | 31.40 | |
| Glycerol nonyl ether | 15.70 | | | | | 31.40 |
| Oleyl alcohol (foam | 2.00 | 2.00 | 2.00 | 2.00 | 2.00 | 2.00 |
| adjuvant) | | | | | | |
| Water, purified, buffered | 34.60 | 34.60 | 48.00 | 66.00 | 34.60 | 34.60 |
| Total | 100 | 100 | 100 | 100 | 100 | 100 |

### b) Stock

| Ingredients for Stock | B7a | B8a | B9a | B10a | B11a | B12a |
|---|---|---|---|---|---|---|
| Ether Solution | (w/w %) | (w/w %) | (w/w %) | (w/w %) | (w/w %) | (w/w %) |
| Glycerol propyl ether | 32.00 | 63.4 | 50.00 | 32.00 | 32.00 | 32.00 |
| Glycerol hexyl ether | 15.70 | | | | 31.40 | |
| Glycerol nonyl ether | 15.70 | | | | | 31.40 |
| Isostearic Acid (foam | 2.00 | 2.00 | 2.00 | 2.00 | 2.00 | 2.00 |
| adjuvant) | | | | | | |
| Water, purified, buffered | 34.60 | 34.60 | 48.00 | 66.00 | 34.60 | 34.60 |
| Total | 100 | 100 | 100 | 100 | 100 | 100 |

### c) 29% to ˜15% Formulations

| Ingredient | W/W % |
|---|---|
| Water | 50.00 |
| Avicel 581RC | 2.00 |
| Polysorbate 80 | 2.00 |
| Stock Ether Solution A Or B | 46.00 |
| Total | 100.00 |
| Propellant (AP-70) | 8.00 |

### d) 61% to ˜31% Formulations

| Ingredient | W/W % |
|---|---|
| Hydroxypropylcellulose EF | 1.00 |
| Polysorbate 80 | 2.00 |
| Stock Ether Solution A Or B | 97.00 |
| Total | 100.00 |
| Propellant (AP-70) | 8.00 |

The compositions may be buffered with a suitable buffer. In the above example the buffer is part of the aqueous phase and is a combination of citric acid (˜0.48% of 100%) and sodium hydroxide (˜0.23% of 100%). A mildly acidic pH within the range of that of skin is preferred.

### B3 Example 6

### Prophetic Vehicle Compositions Containing. Glycerol Propyl Ether; Glycerol Hexyl Ether; Glycerol Nonyl Ether and a Non-Volatile Ether

The following foamable compositions can be prepared as detailed in example 2.

### a) Stock

| Stock Ether | B13 | B14 | B15 | B16 | B17 | B18 |
|---|---|---|---|---|---|---|
| Solution | W/W % | W/W % | W/W % | W/W % | W/W % | W/W % |
| Glycerol propyl | 32.00 | 55.40 | 44.00 | 26.00 | 32.00 | 32.00 |
| ether | | | | | | |
| Glycerol hexyl | 11.70 | | | | 23.40 | |
| ether | | | | | | |
| Glycerol nonyl | 11.70 | | | | | 23.40 |
| ether | | | | | | |
| glycerol, 1,3- | 8.00 | 8.00 | 8.00 | 8.00 | 8.00 | 8.00 |
| dimethyl ether | | | | | | |
| (Non-volatile | | | | | | |
| ether) | | | | | | |
| Water, | 36.60 | 36.60 | 48.00 | 66.00 | 36.60 | 36.60 |
| purified, | | | | | | |
| buffered | | | | | | |
| Total | 100 | 100 | 100 | 100 | 100 | 100 |

b) ˜25% to ˜12%+˜4% non-volatile ether Formulations

| Ingredient | W/W % |
|---|---|
| Water | 50.00 |
| Avicel 581RC | 2.00 |
| Polysorbate 80 | 2.00 |
| Stock Ether Solution | 46.00 |
| Total | 100.00 |
| Propellant (AP-70) | 8.00 |

c) ˜54% to ˜25%+˜8% non-volatile ether Formulations

| Ingredient | W/W % |
|---|---|
| Hydroxypropylcellulose EF | 1.00 |
| Polysorbate 80 | 2.00 |
| Stock Ether Solution | 97.00 |
| Total | 100.00 |
| propellant (AP-70) | 8.00 |

The compositions may be buffered with a suitable buffer. In the above example the buffer is part of the aqueous phase and is a combination of citric acid (˜0.48% of 100%) and sodium hydroxide (˜0.23% of 100%). A mildly acidic pH within the range of that of skin is preferred.

### B4 Example 7

### Prophetic Vehicle Compositions Containing. Glycerol Propyl Ether; Glycerol Hexyl Ether; Glycerol Nonyl Ether a Foam Adjuvant and a Non-Volatile Ether

The following foamable compositions can be prepared as detailed in example 2.

### a) Stock

| Ingredients for Stock | B19 | B20 | B21 | B22 | B23 | B24 |
|---|---|---|---|---|---|---|
| Ether Solution | (w/w %) | (w/w %) | (w/w %) | (w/w %) | (w/w %) | (w/w %) |
| Glycerol propyl ether | 32.00 | 55.40 | 44.00 | 26.00 | 32.00 | 32.00 |
| Glycerol hexyl ether | 11.70 | | | | 23.40 | |
| Glycerol nonyl ether | 11.70 | | | | | 23.40 |
| glycerol, 1,3-dimethyl | 8.00 | 8.00 | 8.00 | 8.00 | 8.00 | 8.00 |
| ether (Non-volatile ether) | | | | | | |
| Isostearic acid (foam | 1-00 | 1-00 | 1-00 | 1-00 | 1-00 | 1-00 |
| adjuvant) | | | | | | |
| Water, purified, buffered | 35.60 | 35.60 | 47.00 | 65.00 | 35.60 | 35.60 |
| Total | 100 | 100 | 100 | 100 | 100 | 100 |

### b) ˜25% to ˜12%+˜4% Non-Volatile Ether Formulations

| Ingredient | W/W % |
|---|---|
| Water | 50.00 |
| Avicel 581RC | 2.00 |
| Polysorbate 80 | 2.00 |
| Stock Ether Solution | 46.00 |
| Total | 100.00 |
| propellant (AP-70) | 8.00 |

### c) ˜54% to ˜25%+˜8% Non-Volatile Ether Formulations

| Ingredient | W/W % |
|---|---|
| Hydroxypropylcellulose EF | 1.00 |
| Polysorbate 80 | 2.00 |
| Stock Ether Solution | 97.00 |
| Total | 100.00 |
| Propellant (AP-70) | 8.00 |

The compositions may be buffered with a suitable buffer. In the above example the buffer is part of the aqueous phase and is a combination of citric acid (˜0.48% of 100%) and sodium hydroxide (˜0.23% of 100%). A mildly acidic pH within the range of that of skin is preferred.

### Section C—Prophetic Pharmaceutical and Cosmetic Formulations

### C1—Example 8

### Exemplary Prophetic Foams Containing Active Pharmaceutical Ingredients (API)

Exemplary concentrations of active ingredients in foamable compositions are set out in Table 2 and in the following additional prophetic examples. Each active ingredient is added into, for example, any of the carriers listed in any of the above Examples in a therapeutically effective concentration and amount. The methodology of addition is well known to those of the art. The composition is adjusted in each case so that it is made up to 100% w/w by addition or reduction of water or ether as is appropriate to the active agents concerned.

### C2—Exemplary Concentration Ranges of Some APIs which are Addable to Foams

TABLE 2

| Class | Concentration | Exemplary Use |
|---|---|---|
| Hydrocortisone | 1% | Steroid responsive inflammation and |
| acetate | | psoriasis or atopic dermatitis |
| Betamethasone | 0.12% | |
| valerate | | |
| Clobetasol | 0.05% | |
| proprionate | | |
| Acyclovir | 5% | Viral infection, herpes |
| Ciclopirox | 1% | Fungal infection, seborrhea, dandruff, |
| Clindamycin | 1-2% | Bacterial infection, acne, rosacea, |
| Azelaic acid | 15% | Acne, rosacea, |
| | | pigmentation disorder and various |
| | | dermatoses |
| Metronidazol | 0.25%-2% | Rosacea, bacterial infections and |
| | | parasite infestations |
| Diclofenac | 1% | Osteoarthritus, joint pain |
| Tacrolimus | 0.2% | Atopic dermatitis, eczema and |
| | | inflammation |
| Caffeine | 5% | anti-cellulite |
| Clotrimazole | 1% | Fungal infection |
| Lidocaine base | 2% | Local anaesthetic |
| Terbinafine HCL | 1% | Fungal infection |
| Gentamycin | 0.1% | Bacterial skin infections, burns or |
| | | ulcers |
| Dexpanthenol | 5% | Wounds, ulcers, minor skin infections |
| Urea | 5-10% | Emollient and keratolytic |
| | | Atopic dermatitis, eczema, ichthyosis |
| | | and hyperkeratotic skin disorders |
| Ammonium | 12%-17.5% | Dry scaly conditions of the skin |
| lactate | | including ichthyosis |
| Povidone-iodine | 10% | Antimicrobial - antiseptic |
| Benzoyl peroxide | 1%-10% | Acne |
| Alpha-hydroxy | 1%-20% | Aging, wrinkles |
| acids | | |
| Salicylic acid | 1%-10% | Acne |
| Hydroquinone | 1%-10% | Pigmentation disorders |
| calcipotriol | 0.005 | Psoriasis |
| Coal tar | 10% | Psoriasis |

### C3—Prophetic Steroid Compositions

The following steroids can be included in carriers, compositions and foams betamethasone valerate 0.12%, clobetasol propionate 0.05%, betamethasone dipropionate 0.05%, fluocinolone acetonide 0.025%, hydrocortisone acetate 0.5% and hydrocortisone butyrate 0.1%.

### C4—Prophetic Vitamin and Steroid Compositions

Additionally, one or more of the following vitamins can be included in the carriers, compositions and foams: vitamin C (ascorbic acid) between 0.1 and 5% e.g., 0.1% 1%, 2% 3%, 4%, or 5%; vitamin C (magnesium ascorbyl phosphate) 3%, retinol 1%, retinoic acid 0.1%, niacinamide 2% and tocopherol 1% and Vitamin K. between 0.1 and 2% e.g., 0.1% or 1% or 2%.

### C5—Prophetic Vitamin and/or Flavenoid Compositions with or without an Additional Therapeutic Agent

Foamable vitamin compositions at either e.g. 0.1% 1%, 2%, 3%, 4%, or 5%, by weight of composition are made up with or without an active agent and added to any of the vehicles or compositions illustrated in the above Examples.

### C6—Different Drug Classes

All the above examples represent different drug classes and it is to be understood that other drugs belonging to each of the classes represented above or described elsewhere in the specification may be included and may be used in the compositions in a safe and effective amount.

What is claimed is:

1. A pharmaceutical or cosmetic vehicle composition comprising:
(a) less than or equal to about 75% by weight of at least one glycerol ether;
(b) at least one stabilizer selected from the group consisting of a surface-active agent, a polymeric agent and mixtures thereof;
(c) water; and
(d) about 3% to about 35% by weight of a liquefied hydrocarbon gas propellant;
wherein the polymeric agent, if present, is about 0.1% to about 5% by weight and is selected from the group consisting of a bioadhesive agent, a gelling agent, a film forming agent and a phase change agent;
wherein the composition is contained in a pressurized container; and
wherein the composition is substantially flowable and provides a breakable and thermolabile foam of fairly good to excellent quality upon release from the container.

2. The composition of claim 1, wherein the composition further comprises a carrier or solvent selected from the group consisting of an organic carrier, a hydrophilic solvent, a hydrophobic solvent, a potent solvent, a polar solvent, a silicone, an emollient, a liquid wax and mixtures thereof;
wherein the organic carrier is selected from the group consisting of mineral oil, a therapeutic oil, triglycerides, medium chain triglyceride (MCT) oil, capric/caprylic triglyceride, cocoglycerides, alkyl esters of fatty acids such as isopropyl palmitate, isopropyl myristate, isopropyl isostearate, poly propylene glycol 15-stearyl ether, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, maleated soybean oil, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxane, polyaryl siloxane, polyalkylaryl siloxane, a polyether siloxane copolymer and a poly(dimethylsiloxane)-(diphenyl-siloxane) copolymer and a polypropylene glycol alkyl ether;
wherein the polar solvent is selected from the group consisting of dimethyl isosorbide, glycerol, propylene glycol, hexylene glycol, diethylene glycol, propylene glycol n-alkanols, terpenes, di-terpenes, tri-terpenes, limonene, terpene-ol, 1-menthol, dioxolane, ethylene glycol, other glycols, oleyl alcohol, alpha-hydroxy acids, such as lactic acid and glycolic acid, sulfoxides, such as dimethylsulfoxide (DMSO), dimethylformanide, methyl dodecyl sulfoxide, dimethylacetamide, azone (1-dodecylazacycloheptan-2-one), 2-(n-nonyl)-1,3-dioxolane, alkanols, such as dialkylamino acetates, and admixtures thereof;
wherein the silicone is selected from the group consisting of a volatile silicone and non-volatile silicone; and
wherein the liquid wax is selected from the group consisting of isostearic acid, oleyl alcohol, and capric alcohol, capryl alcohol, isostearic acid, caprylic acid, caproic acid, and butyric acid, jojoba oil.

3. The composition of claim 1 wherein the surface-active agent is a liquid or a combination comprising a solid and a liquid.

4. The composition of claim 1, wherein the surface active agent is selected from the group consisting of a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59, a polyoxyethylene alkylyl ether, polyoxyethylene cetyl ether, polyoxyethylene palmityl ether, polyethylene oxide hexadecyl ether, polyethylene glycol cetyl ether, brij 38, brij 52, brij 56 and brij W1, a sucrose ester, a partial ester of sorbitol, sorbitan monolaurate, sorbitan monolaurate a monoglyceride, a diglyceride, isoceteth-20, a sucrose ester, or selected from the group consisting of steareth 2, glyceryl monostearate/PEG 100 stearate, Glyceryl Stearate, Steareth-21, peg 40 stearate, polysorbate 40, polysorbate 60, polysorbate 80, sorbitan stearate, laureth 4, Sorbitan monooleate, ceteareth 20, steareth 20, ceteth 20, Macrogol Cetostearyl Ether, ceteth 2, PEG-30 Dipolyhydroxystearate, sucrose distearate, polyoxyethylene (100) stearate, PEG 100 stearate, PEG 40 stearate, laureth 4, cetomacrogol ether, Cetearyl alcohol, Cetearyl glucoside, Oleyl alcohol, Steareth-2, Diisopropyl adipate, Capric/caprilic triglicerides, Polysorbate 20; Montanov 68 (cetearyl alcohol and cetearyl glucoside), Sharonmix 824 (a liquid blend of methyl paraben, ethyl paraben and propyl paraben—in phenoxyethanol), Simusol 165 (Glyceryl stearate and PEG-100 stearate), methyl glucose sequistearate, PEG 30 dipolyhydroxystearate, sucrose stearic acid esters, sorbitan laureth, sorbitan stearate and mixtures thereof.

5. The composition of claim 1 wherein the polymeric agent is selected from the group consisting of avicel, carbopol 934, pemulen TR2, klucel EF, xanthan gum, methocel A4M, carboxy methyl cellulose, locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, an amine-bearing polymer, chitosan, alginic acid, hyaluronic acid, a chemically modified starch, a carboxyvinyl polymer, polyvinylpyrrolidone, polyvinyl alcohol, a polyacrylic acid polymer, a polymethacrylic acid polymer, polyvinyl acetate, a polyvinyl chloride polymer, a polyvinylidene chloride polymer, methylcellulose, hydroxypropyl cellulose, hydroxypropyl methylcellulose, hydroxyethyl cellulose, hydroxy propylmethyl cellulose, methylhydroxyethylcellulose, methyl hydroxypropylcellulose, hydroxyethylcarboxymethylcellulose, carboxymethyl cellulose, carboxymethylcellulose carboxymethylhydroxyethylcellulose, a cationic cellulose, PEG 1000, PEG 4000, PEG 6000, PEG 8000, a derivatized polymeric emulsifier, aluminum starch octenylsuccinate (ASOS), sodium starch octenylsuccinate.

6. The composition of claim 1, wherein the glycerol ether is a volatile glycerol ether, and wherein the glycerol ether evaporates upon application to a topical surface at about 37° C.

7. The composition of claim 1, wherein the glycerol ether is selected from the group consisting of glycerol propyl ether, glycerol hexyl ether, glycerol nonyl ether and mixtures thereof.

8. The composition of claim 1, further containing a non-volatile ether.

9. The composition of claim 8, wherein the non-volatile ether is selected from the group consisting of Glycerol, 1(2-chlorophenyl)ether; glycerol, 1-(4-chlorophenyl)ether; glycerol, 1,2-dimethyl ether; glycerol, 1,3-dimethyl ether; glycerol, 1,3-diphenylether; glycerol, 1-(2-methoxyphenyl)ether; glycerol, 1-methyl ether; glycerol, 2-methyl ether; glycerol, 1-octadecyl ether; glycerol, 1-phenyl ether; and polyoxypropylene glyceryl ether.

10. The composition of claim 1, further containing a foam adjuvant.

11. The composition of claim 1, wherein the ratio of glycerol ether to stabilizer and water ranges from about 9:1 to about 1:100.

12. The composition of claim 11, wherein the ratio of glycerol ether to stabilizer and water ranges from about 5:1 to about 1:2.

13. The composition of claim 1, further comprising an additional component selected from the group consisting of a modulating agent, a polar solvent, an antiperspirant, an anti-static agent, a buffering agent, a bulking agent, a chelating agent, a colorant, a conditioner, a deodorant, a diluent, a dye, an emollient, fragrance, a humectant, an occlusive agent, a penetration enhancer, a perfuming agent, a permeation enhancer, a pH-adjusting agent, a preservative, a skin penetration enhancer, a sunscreen, a sun blocking agent, a sunless tanning agent, an antioxidant, an antiseptive, a flavonoid, and a vitamin.

14. A therapeutic composition comprising:
i. at least one volatile glycerol ether;
ii. at least one stabilizer selected from the group consisting of a surface-active agent, a polymeric agent and mixtures thereof;
iii. water;
iv. a pharmaceutical or cosmetic agent homogeneously distributed in the composition; and
v. about 3% to about 35% by weight of a liquefied hydrocarbon gas propellant
wherein the polymeric agent, if present is about 0.1% to about 5% by weight and is selected from the group consisting of a bioadhesive agent, a gelling agent, a film forming agent and a phase change agent;
wherein the volatile glycerol ether evaporates upon application to a topical surface at about 37° C. to deposit the pharmaceutical or cosmetic agent on the target topical surface; and
wherein the composition is contained in a pressurized container; and
wherein the composition is substantially flowable and provides a breakable and thermolabile foam of fairly good to excellent quality upon release from the container.

15. The therapeutic composition of claim 14, wherein the pharmaceutical or cosmetic agent is selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, coal tar, dicarboxylic acids, disinfectants, fungicides, hair growth regulators, hormones, hydroxy acids, interferons, immunosuppressants, immunoregulating agents, insecticides, insect repellents, keratolytic agents, lactams, metals, metal oxides, mitocides, neuropeptides, steroids, non-steroidal anti-inflammatory agents, oxidizing agents, pediculicides, photodynamic therapy agents, phosphcholines, alkyl phosphoric acids, retinoids, sanatives, scabicides, self tanning agents, skin whitening agents, vasoconstrictors, vasodilators, vitamins, vitamin D derivatives, wound healing agents and wart removers as well as agents having activity against skin and topical lesions, leishmaniasis, superficial basal cell carcinomas, actinic keratoses, Bowen's disease and/or other squamous cell carcinomas and molluscum contagiosum.

16. The foamable therapeutic composition of claim 14, wherein the volatile glycerol ether is present in the composition in an amount sufficient to solubilize the active agent.

17. The foamable therapeutic composition of claim 14, wherein the active agent is a phosphocholine or an alkyl phosphoric acid.

18. The foamable therapeutic composition of claim 14, wherein the active agent is hexadecylphosphocholine.

19. The foamable therapeutic composition of claim 14, wherein the active agent is effective against any one or more of skin or topical lesions, cancer, skin cancer and leishmaniasis.

20. The foamable therapeutic composition of claim 17 further comprising at least one excipient compound selected from the group consisting of a petrolatum, a therapeutic oil, a liquid wax, a liquid emollient and mixtures thereof.

21. The composition of claim 14, wherein the composition further comprises a carrier or solvent selected from the group consisting of an organic carrier, a hydrophilic solvent; a hydrophobic solvent; a potent solvent; a polar solvent, a silicone, an emollient, a liquid wax and mixtures thereof.

22. The composition of claim 14, wherein the glycerol ether, stabilizer and water are selected to generate a single phase formulation or substantially homogenous suspension.

23. The composition of claim 14, wherein the glycerol ether, stabilizer and water are selected to generate an emulsion that is substantially resistant to phase reversal.

24. A method of treating, ameliorating or preventing a disorder of a mammalian subject, the method comprising:
administering a foamable therapeutic composition to a target site, the composition comprising:
a. a therapeutically effective amount of a pharmaceutical or cosmetic agent substantially homogenously distributed in the glycerol ether composition;
b. less than or equal to about 75% by weight of at least one glycerol ether;
c. at least one stabilizer selected from the group consisting of a surface-active agent, a polymeric agent and mixtures thereof;
d. about 3% to about 35% by weight of a liquefied hydrocarbon gas propellant and
wherein the polymeric agent is about 0.1% to about 5% by weight and is selected from the group consisting of a bioadhesive agent, a gelling agent, a film forming agent and a phase change agent;
wherein the composition is contained in a pressurized container; and
wherein the composition is substantially flowable and provides a breakable and thermolabile foam of fairly good to excellent quality upon release from the container; and
wherein the glycerol ether evaporates upon application to a topical surface at about 37° C. to deposit the pharmaceutical or cosmetic agent on the target topical surface.

25. The method of claim 24, wherein the target site is selected from the group consisting of the skin, a body cavity, a mucosal surface, the ear canal, the vagina and the rectum.

26. The method of claim 24, wherein the disorder is selected from the group consisting of dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, eethyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, granuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo; and wherein the active agent is suitable for treating said disorderm or is selected from the group consisting of chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranuloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, topical or skin lesions, leishmaniosis, cancer, skin cancer, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; warts; herpes simplex virus infections, other viral infections, skin and topical lesions, leishmaniasis, superficial basal cell carcinomas, actinic keratoses, Bowen's disease and/or other squamous cell carcinomas, molluscum contagiosum and eczema, and wherein the active agent is suitable for treating said disorder.

27. The method of claim 24, wherein the disorder is a dermatological disorder which can be treated, ameliorated or prevented by a topical phosphocholine or alkyl phosphoric acid.

28. The method of claim 24 wherein the active agent is selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, coal tar, dicarboxylic acids, disinfectants, fungicides, hair growth regulators, hormones, hydroxy acids, interferons, immunosuppressants, immunoregulating agents, insecticides, insect repellents, keratolytic agents, lactams, metals, metal oxides, mitocides, neuropeptides, steroids, non-steroidal anti-inflammatory agents, oxidizing agents, pediculicides, photodynamic therapy agents, phosphcholines, alkyl phosphoric acids, retinoids, sanatives, scabicides, self tanning agents, skin whitening agents, vasoconstrictors, vasodilators, vitamins, vitamin D derivatives, wound healing agents and wart removers as well as agents having activity against skin and topical lesions, leishmaniasis, superficial basal cell carcinomas, actinic keratoses, Bowen's disease and/or other squamous cell carcinomas and molluscum contagiosum.

29. The method of claim 24 wherein the glycerol ether is a volatile glycerol ether, wherein the volatile glycerol ether evaporates upon application to a topical surface at about 37° C.

30. A pharmaceutical composition comprising:
i. a liquid volatile glycerol ether
ii. a stabilizer comprising about 1% to about 5% of at least one surface-active agent and about 0.1% to about 5% of at least one polymeric agent selected from the group consisting of a bioadhesive agent, a gelling agent, a film forming agent and a phase change agent;
iii. water;
iv. an active agent substantially homogenously distributed in the composition; and
v. 3% to about 35% by weight of a liquefied hydrocarbon gas propellant
wherein the volatile glycerol ether evaporates upon application to a topical surface at about 37° C. to deposit the pharmaceutical or cosmetic agent for absorption onto a topical surface; and
wherein the composition is contained in a pressurized container; and
wherein the composition provides a breakable and thermolabile foam of about fairly good to about excellent quality upon release from the container.

31. The pharmaceutical composition of claim 30, wherein a liquid volatile glycerol ether comprises about 1% to about 75% by weight of a volatile glycerol ether selected from the group consisting of glycerol propyl ether, glycerol hexyl ether and glycerol nonyl ether and mixtures thereof.

32. The composition of claim 1 in a non-foam state.

33. A pharmaceutical or cosmetic vehicle composition comprising:
(a) less than or equal to about 75% by weight of at least one glycerol ether;
(b) at least one stabilizer selected from the group consisting of a surface-active agent, a polymeric agent and mixtures thereof; and
(c) water;
wherein the polymeric agent, if present, is about 0.1% to about 10% by weight of and is selected from the group consisting of a bioadhesive agent, a gelling agent, a film forming agent and a phase change agent;
wherein the composition is contained in a pressurized container; and
wherein the composition is substantially flowable and provides a breakable and thermolabile foam of about fairly good to about excellent quality upon release from the container; and
wherein the composition is able to deposit a pharmaceutical or cosmetic agent for absorption onto a topical surface.
